(19) United States
(12) Patent Application Publication
(10) Pub. No.: US 2020/0002307 A1
(43) Pub. Date: Jan. 2, 2020

(54) METHOD FOR PURIFYING COMPOUND OR RESIN AND METHOD FOR PRODUCING COMPOSITION

(71) Applicant: Mitsubishi Gas Chemical Company, Inc., Tokyo (JP)

(72) Inventors: Naoya UCHIYAMA, Kurashiki-shi, Okayama (JP); Junya HORIUCHI, Hiratsuka-shi, Kanagawa (JP); Takashi MAKINOSHIMA, Hiratsuka-shi, Kanagawa (JP); Masatoshi ECHIGO, Chiyoda-ku, Tokyo (JP)

(21) Appl. No.: 16/489,258

(22) PCT Filed: Feb. 28, 2018
(86) PCT No.: PCT/JP2018/007609

§ 371 Date: Aug. 27, 2019

(30) Foreign Application Priority Data
Feb. 28, 2017 (JP) 2017-037388

(51) Int. Cl. C07D 311/82; C07D 311/78; C08G 8/20; C07D 405/04; C07C 39/15; C08G 8/04; C07C 39/14

(52) U.S. Cl. CPC C07D 311/82; C07D 311/78; C08G 8/20; C07C 2601/14; C07C 39/15; C08G 8/04; C07C 39/14; C07D 405/04

(57) ABSTRACT

A method for purifying a material, the method comprising:
a step of preparing a solution comprising a solvent and at least one material selected from the group consisting of a compound represented by the following formula (1A) and a resin having a structure represented by the following formula (2A); and
a step of purification in which the solution is passed through a filter:

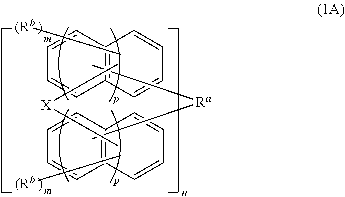

wherein, X represents an oxygen atom, a sulfur atom, a single bond, or non-crosslinked state; R^{a} represents a 2n-valent group having 1 to 40 carbon atoms or a single bond; each R^{b} independently represents one of various functional groups; each m is independently an integer of 0 to 9; n is an integer of 1 to 4; and each p is independently an integer of 0 to 2; provided that at least one R^{b} represents a group comprising one selected from a hydroxyl group and a thiol group, and all m cannot be 0 at the same time;

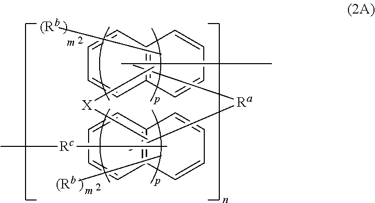

wherein, X, R^{a}, R^{b}, n and p are the same as defined in the formula (1A); R^{c} represents a single bond or an alkylene group having 1 to 40 carbon atoms; each m² is independently an integer of 0 to 8; provided that at least one R^{b} represents a group comprising one or more selected from a hydroxyl group and a thiol group, and all m² cannot be 0 at the same time.

### TECHNICAL FIELD

The present invention relates to a method for purifying a compound or resin having a specific structure and a method for producing a composition.

### BACKGROUND ART

A polyphenol compound or resin having a specific backbone described in Patent Documents 1 and 2 is excellent in heat resistance, etching resistance and solvent solubility, and therefore is used for semiconductor coating agents, resist materials and semiconductor underlayer film formation materials.

### CITATION LIST

### Patent Document

Patent Document 1: International Publication No. WO2013/024778
Patent Document 2: International Publication No. WO2013/024779

### SUMMARY

### Technical Problem

In the above applications, in particular, the metal content is an important performance item for an enhancement in yield. That is, when a polyphenol compound or resin having a specific backbone, high in the metal content, is used, the metal remains in a semiconductor to result in a reduction in electrical properties of the semiconductor, and therefore a reduction in the metal content is demanded.

As a method for purifying a polyphenol compound or resin having a specific backbone to reduce the metal content therein, there is considered a method comprising subjecting to recrystallization by adding an ion-exchange water or pure water to a mixture including the compound or resin and an organic solvent and then subjecting to solid-liquid separation; or a method comprising dissolving the compound or resin in an organic solvent optionally immiscible with water and bring the solution into contact with an aqueous solution to perform an extraction treatment, thereby transferring the metal components to an aqueous phase, and thereafter separating an organic phase and an aqueous phase to reduce the metal content; or the like.

When the polyphenol compound or resin having a specific backbone, high in the metal content, is used as a raw material, however, the above-described method has the problem of having insufficient removal effect for specific metal species.

As an alternative method, there are also considered a method of bringing a mixture including the compound or resin and an organic solvent into contact with an ion-exchange resin. If various metal ions are contained, however, the method using an ion-exchange resin has the problem of having difficulty in selection of the ion-exchange resin and thus having difficulty in removal of the metal ions depending on the kinds of the metals, the problem of having difficulty in removal of a nonionic metal, and also the problem of being large in running cost.

An object of the present invention is to provide a purification method that enables to significantly reduce the contents of various metal components included in a compound or resin having a specific structure.

### Solution to Problem

The present inventors have intensively studied in order to solve the above problems, and as a result, have found that a solution including a compound or resin having a specific structure and a solvent is passed through a filter to result in a significant reduction in the contents of metal components in the solution, thereby leading to the present invention.

That is, the present invention is as follows.

[1]

A method for purifying a material, the method comprising:

a step of preparing a solution comprising a solvent and at least one material selected from the group consisting of a compound represented by the following formula (1A) and a resin having a structure represented by the following formula (2A); and

a step of purification in which the solution is passed through a filter:

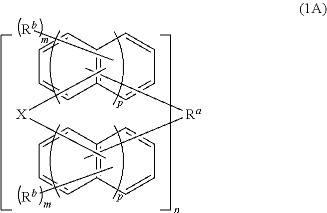

wherein, X represents an oxygen atom, a sulfur atom, a single bond, or non-crosslinked state; R^{a} represents a 2n-valent group having 1 to 60 carbon atoms or a single bond; each R^{b} independently represents an optionally substituted alkyl group having 1 to 40 carbon atoms, an optionally substituted aryl group having 6 to 40 carbon atoms, an optionally substituted alkenyl group having 2 to 40 carbon atoms, an optionally substituted alkoxy group having 1 to 40 carbon atoms, a halogen atom, a thiol group or a hydroxyl group; each m is independently an integer of 0 to 9; n is an integer of 1 to 4; and each p is independently an integer of 0 to 2; provided that at least one R^{b} represents a group comprising one selected from a hydroxyl group and a thiol group, and all m cannot be 0 at the same time.

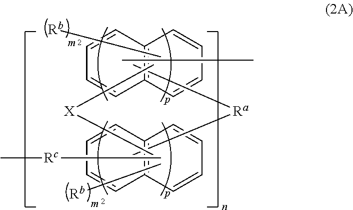

wherein, X, R^{a}, R^{b}, n and p are the same as defined in the formula (1A); R^{c} represents a single bond or an alkylene group having 1 to 40 carbon atoms; each m² is independently an integer of 0 to 8; provided that at least one R^{b} represents a group comprising one or more selected from a hydroxyl group and a thiol group, and all m² cannot be 0 at the same time.

[2]

The method for purifying the material according to [1], wherein the purification is performed in an atmosphere with an oxygen concentration of less than 20%.

[3]

The method for purifying the material according to [1] or [2], wherein the filter has a nominal pore size of 0.2 μm or less.

[4]

The method for purifying the material according to any of [1] to [3], wherein the filter is one or more selected from the group consisting of a hollow fiber membrane filter, a membrane filter and a pleated membrane filter.

[5]

The method for purifying the material according to any of [1] to [4], wherein the filter is made of one or more filter media selected from the group consisting of a polyamide, a polyolefin resin and a fluorocarbon resin.

[6]

The method for purifying the material according to any of [1] to [5], wherein the filter comprises an ion exchanger.

[7]

The method for purifying the material according to any of [1] to [6], wherein the filter comprises a material having a zeta potential.

[8]

The method for purifying the material according to any of [1] to [7], wherein the solvent is one or more selected from the group consisting of ethyl acetate, butyl acetate, methyl isobutyl ketone, propylene glycol monomethyl ether, propylene glycol monomethyl ether acetate, cyclopentanone and cyclohexanone.

[9]

The method for purifying the material according to any of [1] to [8], wherein a content of chromium among metals comprised in the solution after the purification is 50 ppb or less based on a mass of the material.

[10]

The method for purifying the material according to any of [1] to [9], wherein the compound represented by the formula (1A) and the resin having a structure represented by the formula (2A) are a compound represented by the following formula (1A′) and a resin having a structure represented by the following formula (2A′), respectively:

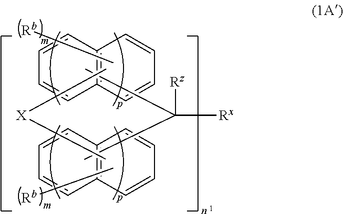

wherein, R^{b}, X, m and p are the same as defined in the formula (1A); R^{x} represents an n-valent group having 1 to 40 carbon atoms or a single bond; R^{z} represents a hydrogen atom, an alkyl group having 1 to 30 carbon atoms or an aryl group having 6 to 30 carbon atoms; and n¹ is an integer of 1 to 4;

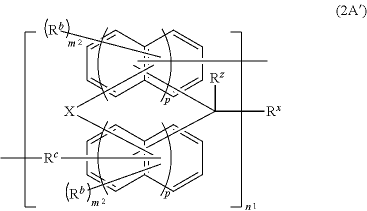

wherein, R^{b}, X, m² and p are the same as defined in the formula (2A); and R^{x}, R^{z} and n¹ are the same as defined in the formula (1A′).

[11]

The method for purifying the material according to any of [1] to [10], wherein the compound represented by the formula (1A) is a compound represented by the formula (1):

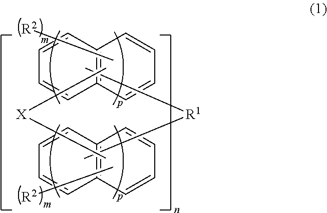

wherein, X, m, n and p are the same as defined in the formula (1A); R¹ is the same as R^{a} defined in the formula (1A); and each R² independently represents an alkyl group having 1 to 40 carbon atoms, an aryl group having 6 to 40 carbon atoms, an alkenyl group having 2 to 40 carbon atoms, an alkoxy group having 1 to 40 carbon atoms, a halogen atom, a thiol group or a hydroxyl group; provided that at least one R² represents one selected from a hydroxyl group and a thiol group, and all m cannot be 0 at the same time.

[12]

The method for purifying the material according to [11], wherein the compound represented by the formula (1) is a compound represented by the following formula (1-1):

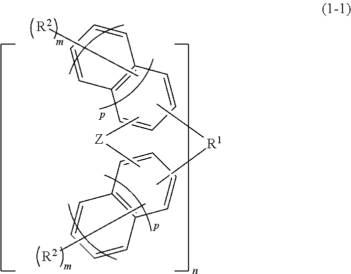

wherein, Z represents an oxygen atom or a sulfur atom; R¹, R², m, p and n are the same as defined in the formula (1); provided that at least one R² represents one selected from a hydroxyl group and a thiol group, and all m cannot be 0 at the same time.

[13]

The method for purifying the material according to [12], wherein the compound represented by the formula (1-1) is a compound represented by the following formula (1-2):

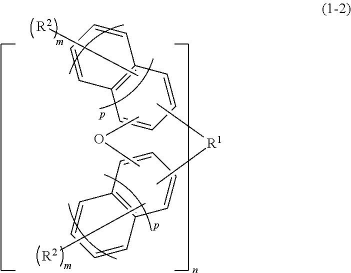

wherein, R¹, R², m, p and n are the same as defined in the formula (1); provided that at least one R² represents one selected from a hydroxyl group and a thiol group, and all m cannot be 0 at the same time.

[14]

The method for purifying the material according to [13], wherein the compound represented by the formula (1-2) is a compound represented by the following formula (1-3):

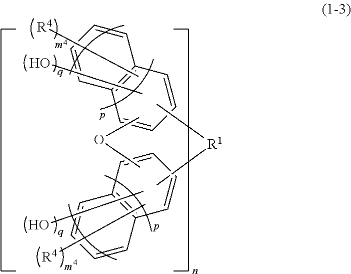

wherein, R¹, p and n are the same as defined in the formula (1); each R⁴ independently represents an alkyl group having 1 to 40 carbon atoms, an aryl group having 6 to 40 carbon atoms, an alkenyl group having 2 to 40 carbon atoms, an alkoxy group having 1 to 40 carbon atoms, a halogen atom or a thiol group; each m⁴ is independently an integer of 0 to 8; and each q is independently an integer of 0 to 8; provided that all q cannot be 0 at the same time).

[15]

The method for purifying the material according to [14], wherein the compound represented by the formula (1-3) is a compound represented by the following formula (1-4):

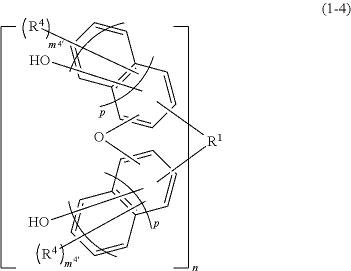

wherein, R¹, p and n are the same as defined in the formula (1); R⁴ is the same as defined in the formula (1-3); and each m^{4′} is independently an integer of 0 to 7.

[16]

The method for purifying the material according to [15], wherein the compound represented by the formula (1-4) is a compound represented by the following formula (1-5):

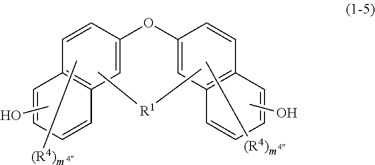

wherein, R¹ is the same as defined in the formula (1); R⁴ is the same as defined in the formula (1-3); and each m^{4″} is independently an integer of 0 to 5.

[17]

The method for purifying the material according to any of [1] to [10], wherein the compound represented by the formula (1A) is a compound represented by the following formula (3):

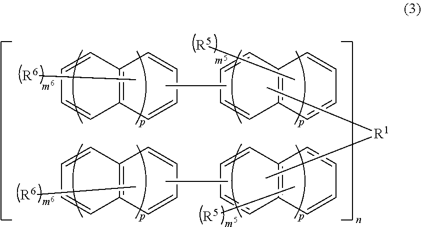

wherein, R¹ is the same as R^{a} defined in the formula (1A); n and p are the same as defined in the formula (1A); R⁵ and R⁶ each independently represent an alkyl group having 1 to 40 carbon atoms, an aryl group having 6 to 40 carbon atoms, an alkenyl group having 2 to 40 carbon atoms, an alkoxy group having 1 to 40 carbon atoms, a halogen atom, a thiol group or a hydroxyl group; each m⁵ is independently an integer of 0 to 8; and each m⁶ is independently an integer of 0 to 9; provided that at least one selected from R⁵ and R⁶ represents one selected from a hydroxyl group and a thiol group, and all of m⁵ and m⁶ cannot be 0 at the same time.

[18]

The method for purifying the material according to [17], wherein the compound represented by the formula (3) is a compound represented by the following formula (3-1):

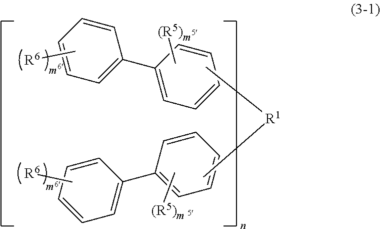

wherein, R¹, R⁵, R⁶ and n are the same as defined in the formula (3); each m^{5′} is independently an integer of 0 to 4; and each m^{6′} is independently an integer of 0 to 5; provided that at least one selected from R⁵ and R⁶ represents one selected from a hydroxyl group and a thiol group, and all of m^{5′} and m^{6′} cannot be 0 at the same time.

[19]

The method for purifying the material according to [18], wherein the compound represented by the formula (3-1) is a compound represented by the following formula (3-2):

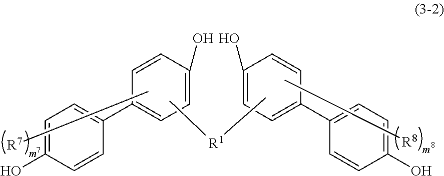

wherein, R¹ is the same as defined in the formula (3); R⁷ and R⁸ each independently represent an alkyl group having 1 to 40 carbon atoms, an aryl group having 6 to 40 carbon atoms, an alkenyl group having 2 to 40 carbon atoms, an alkoxy group having 1 to 40 carbon atoms, a halogen atom, a thiol group or a hydroxyl group; m⁷ and m⁸ each are independently an integer of 0 to 7.

[20]

The method for purifying the material according to any of [1] to [19], wherein the resin having a structure represented by the formula (2A) is a resin having a structure represented by the following formula (2):

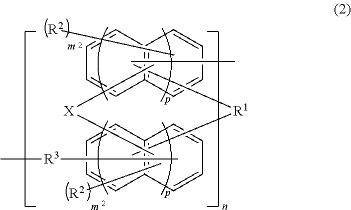

wherein, X, R¹, R², n and p are the same as defined in the formula (1); R³ is the same as R^{c} defined in the formula (2A); and m² is the same as defined in the formula (2A); provided that at least one R² represents one selected from a hydroxyl group and a thiol group, and all m² cannot be 0 at the same time.

[21]

The method for purifying the material according to [20], wherein the resin having a structure represented by the formula (2) is a resin having a structure represented by the following formula (2-1):

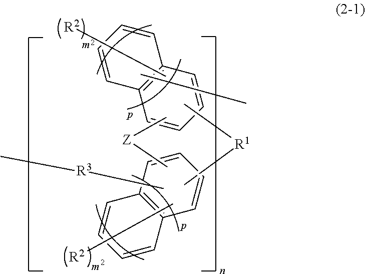

wherein, Z is the same as defined in the formula (1-1); R¹, R², R³, m², p and n are the same as defined in the formula (2); provided that at least one R² represents one selected from a hydroxyl group and a thiol group, and all m² cannot be 0 at the same time.

[22]

The method for purifying the material according to any of [1] to [19], wherein the resin having a structure represented by the formula (2A) is a resin having a structure represented by the following formula (4):

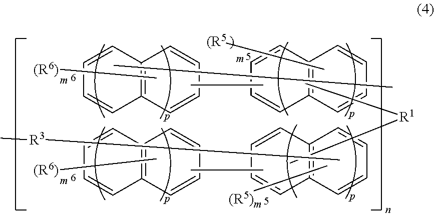

wherein, R¹, R⁵, R⁶, m⁵, m⁶, p and n are the same as defined in the formula (3); and R³ is the same as defined in the formula (2); provided that at least one selected from R⁵ and R⁶ represents one selected from a hydroxyl group and a thiol group, and all of m⁵ and m⁶ cannot be 0 at the same time.

[23]

A method for producing a composition comprising one or more materials selected from the group consisting of a compound represented by the following formula (1A) and an resin having a structure represented by the following formula (2A), 99 ppb or less of Na, less than 60 ppb of Fe, less than 80 ppb of Cr and less than 70 ppb of Sn, the method comprising:

a step of preparing a solution comprising a solvent and a precursor composition comprising the material and more than 99 ppb of Na, 60 ppb or more of Fe, 80 ppb or more of Cr and 70 ppb or more of Sn; and

a step of passing the solution through a filter to thereby reduce contents of Na, Fe, Cr and Sn in the solution to 99 ppb or less, less than 60 ppb, less than 80 ppb and less than 70 ppb, respectively:

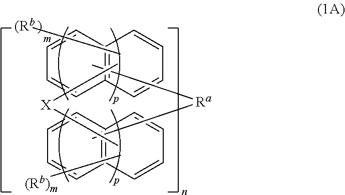

wherein, X represents an oxygen atom, a sulfur atom, a single bond, or non-crosslinked state; R^{a} represents a 2n-valent group having 1 to 60 carbon atoms or a single bond; each R^{b} independently represents an optionally substituted alkyl group having 1 to 40 carbon atoms, an optionally substituted aryl group having 6 to 40 carbon atoms, an optionally substituted alkenyl group having 2 to 40 carbon atoms, an optionally substituted alkoxy group having 1 to 40 carbon atoms, a halogen atom, a thiol group or a hydroxyl group; each m is independently an integer of 0 to 9; n is an integer of 1 to 4; and each p is independently an integer of 0 to 2; provided that at least one R^{b} represents a group comprising one selected from a hydroxyl group and a thiol group, and all m cannot be 0 at the same time;

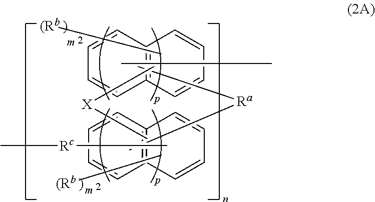

wherein, X, R^{a}, R^{b}, n and p are the same as defined in the formula (1A); R^{c} represents a single bond or an alkylene group having 1 to 40 carbon atoms; each m² is independently an integer of 0 to 8; provided that at least one R^{b} represents a group comprising one or more selected from a hydroxyl group and a thiol group, and all m² cannot be 0 at the same time.

### Advantageous Effects of Invention

According to the present invention, the contents of various metal components in a compound or resin having a specific structure can be significantly reduced.

### DESCRIPTION OF EMBODIMENTS

Hereinafter, an embodiment of the present invention (hereinafter, referred to as “present embodiment”) will be described, but the present invention is not limited thereto and various variations can be made without departing from the scope of the invention.

A method for purifying a material according to the present embodiment comprises: a step of preparing a solution comprising one or more materials selected from a compound represented by the following formula (1A) and a resin having a structure represented by the following formula (2A) and a solvent; and a step of purification in which the solution is passed through a filter.

According to the method for purifying a material according to the present embodiment, which is configured as described above, the contents of various metal components in the material can be significantly reduced.

Herein, the term “purification” in the present embodiment means an operation to sufficiently reduce the metal components that can coexist with the material, and in the material after purification, specifically, the Na content is 99 ppb or less of Na, the Fe content is less than 60 ppb, the Cr content is less than 80 ppb of Cr and the Sn content is less than 70 ppb. In the present embodiment, it is preferable that for the contents of the metal components which can coexist with the material after purification, the Na content is 50 ppb or less, the Fe content is 50 ppb or less, and the Cr content is 50 ppb or less and the Sn content is 50 ppb or less. The contents of these metal components can be measured by the method described in the Examples described hereinbelow.

Herein, the term “passed through” means that the above-described solution is passed from the outside of the filter through the inside of the filter and is allowed to move out of the filter again. For example, a mode in which the solution is simply brought into contact with the surface of the filter and a mode in which the solution is brought into contact on the surface while being allowed to move outside an ion-exchange resin (that is, a mode in which the solution is simply brought into contact) are excluded.

### [Compound Represented by Formula (1A)]

The compound used in the present embodiment is a compound represented by the following formula (1A).

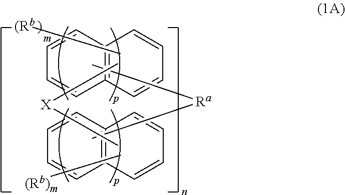

In the formula (1A), X represents an oxygen atom, a sulfur atom, a single bond, or non-crosslinked state.

R^{a} represents a 2n-valent group having 1 to 60 carbon atoms or a single bond. The term “2n-valent group having 1 to 60 carbon atoms” refers to, for example, an alkylene group having 1 to 60 carbon atoms when n=1, an alkanetetrayl group having 1 to 60 carbon atoms when n=2, an alkanehexayl group having 2 to 60 carbon when n=3, and an alkaneoctayl group having 3 to 60 carbon when n=4. Examples of the 2n-valent group include a group having a linear hydrocarbon group, a branched hydrocarbon group or an alicyclic hydrocarbon group. Herein, the alicyclic hydrocarbon group also includes a bridged alicyclic hydrocarbon.

The 2n-valent group may also include a halogen group, a nitro group, an amino group, a hydroxyl group, an alkoxy group, a thiol group or an aromatic group having 6 to 40 carbon atoms. In addition, the 2n-valent group may include an ether bond, a ketone bond, an ester bond or a double bond.

Furthermore, the carbon number is preferably 1 to 40.

Each R^{b} independently represents an optionally substituted alkyl group having 1 to 40 carbon atoms, an optionally substituted aryl group having 6 to 40 carbon atoms, an optionally substituted alkenyl group having 2 to 40 carbon atoms, an optionally substituted alkoxy group having 1 to 40 carbon atoms, a halogen atom, a thiol group or a hydroxyl group. Herein, the alkyl group may be linear, branched or cyclic.

Herein, at least one R^{b} represents a group comprising one selected from a hydroxyl group and a thiol group.

Each m is independently an integer of 0 to 9. Herein, all q cannot be 0 at the same time.

n is an integer of 1 to 4; and each p is independently an integer of 0 to 2.

The compound represented by the formula (1A) is preferably a compound represented by the following formula (1) from the viewpoint of ease of production.

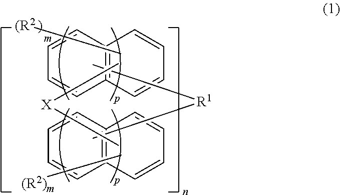

In the formula (1), X, m, n and p are the same as defined above. Herein, all m cannot be 0 at the same time. R¹ is the same as R^{a} defined above.

Each R² independently represents an alkyl group having 1 to 40 carbon atoms, an aryl group having 6 to 40 carbon atoms, an alkenyl group having 2 to 40 carbon atoms, an alkoxy group having 1 to 40 carbon atoms, a halogen atom, a thiol group or a hydroxyl group. Herein, the alkyl group may be linear, branched or cyclic.

Herein, at least one R² represents one selected from a hydroxyl group and a thiol group.

The compound represented by the formula (1) is preferably a compound represented by the following formula (1-1) from the viewpoint of heat resistance.

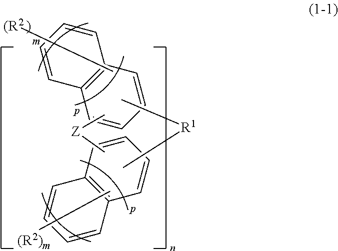

In the formula (1-1), Z represents an oxygen atom or a sulfur atom, and R¹, R², m, p and n are the same as defined in the formula (1). Herein, all m cannot be 0 at the same time, and at least one R² represents one selected from a hydroxyl group and a thiol group.

The compound represented by the formula (1-1) is preferably a compound represented by the following formula (1-2) from the viewpoint of supply of raw materials.

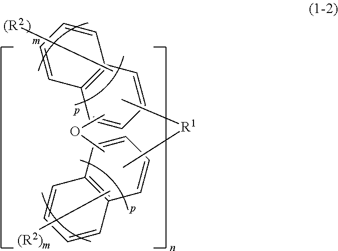

In the formula (1-2), R¹, R², m, p and n are the same as defined in the formula (1). Herein, all m cannot be 0 at the same time, and at least one R² represents one selected from a hydroxyl group and a thiol group.

Furthermore, the compound represented by the formula (1-2) is preferably a compound represented by the following formula (1-3) from the viewpoint of thermosetting properties and dissolution stability.

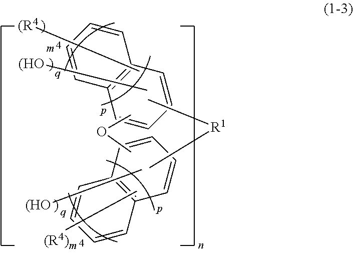

In the formula (1-3), R¹, p and n are the same as defined in the formula (1). Each R⁴ independently represents an alkyl group having 1 to 40 carbon atoms, an aryl group having 6 to 40 carbon atoms, an alkenyl group having 2 to 40 carbon atoms, an alkoxy group having 1 to 40 carbon atoms, a halogen atom or a thiol group. Herein, the alkyl group may be linear, branched or cyclic.

Each m⁴ is independently an integer of 0 to 8; and each q is independently an integer of 0 to 8. Herein, all q cannot be 0 at the same time.

Furthermore, the compound represented by the formula (1-3) is preferably a compound represented by the following formula (1-4) from the viewpoint of heat resistance and dissolution stability.

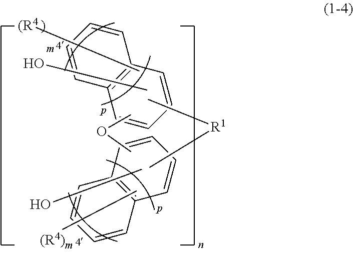

In the formula (1-4), R¹, p and n are the same as defined in the formula (1). R⁴ is the same as defined in the formula (1-3).

Each m^{4′} is independently an integer of 0 to 7.

Furthermore, the compound represented by the formula (1-4) is preferably a compound represented by the following formula (1-5) from the viewpoint of availability of raw materials and ease of production.

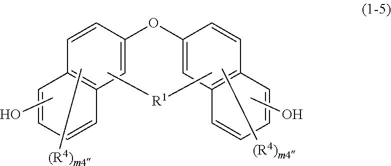

In the formula (1-5), R¹ is the same as defined in the formula (1) and R⁴ is the same as defined in the formula (1-3). Each m^{4″} is independently an integer of 0 to 5.

In addition, in the above formula (1-5), R¹ preferably has at least one hydrogen atom or methyl group.

Furthermore, the compound represented by the formula (1A) is preferably a compound represented by the following formula (3) from the viewpoint of improvement in solubility.

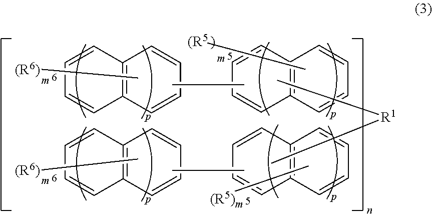

In the formula (3), n and p are the same as defined in the formula (1A); R¹ is the same as R^{a} defined in the formula (1A); and R⁵ and R⁶ each independently represent an alkyl group having 1 to 40 carbon atoms, an aryl group having 6 to 40 carbon atoms, an alkenyl group having 2 to 40 carbon atoms, an alkoxy group having 1 to 40 carbon atoms, a halogen atom, a thiol group or a hydroxyl group. Herein, the alkyl group may be linear, branched or cyclic.

Each m⁵ is independently an integer of 0 to 8; and each m⁶ is independently an integer of 0 to 9. Herein, at least one selected from R⁵ and R⁶ represents one selected from a hydroxyl group and a thiol group, and all of m⁵ and m⁶ cannot be 0 at the same time.

The compound represented by the formula (3) is preferably a compound represented by the following formula (3-1) from the viewpoint of availability of raw materials.

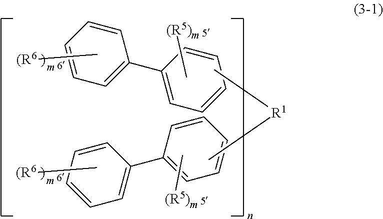

In the formula (3-1), R¹, R⁵, R⁶ and n are the same as defined in the formula (3). Each m^{5′} is independently an integer of 0 to 4; and each m^{6′} is independently an integer of 0 to 5. Herein, at least one selected from R⁵ and R⁶ represents one selected from a hydroxyl group and a thiol group, and all of m^{5′} and m^{6′} cannot be 0 at the same time.

The compound represented by the formula (3-1) is preferably a compound represented by the following formula (3-2) from the viewpoint of availability of raw materials and ease of production.

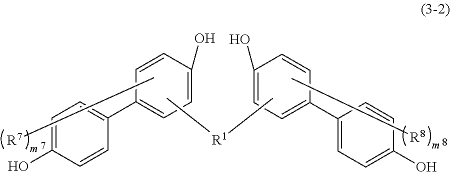

In the formula (3-2), R¹ is the same as defined in the formula (3). R⁷ and R⁸ each independently represent a linear, branched or cyclic alkyl group having 1 to 40 carbon atoms, an aryl group having 6 to 40 carbon atoms, an alkenyl group having 2 to 40 carbon atoms, an alkoxy group having 1 to 40 carbon atoms, a halogen atom, a thiol group or a hydroxyl group. Herein, the alkyl group may be linear, branched or cyclic.

m⁷ and m⁸ each are independently an integer of 0 to 7.

In the above formula (3-2), R¹ preferably has at least one hydrogen atom or methyl group.

### [Compound Represented by Formula (1A′)]

In the present embodiment, the compound represented by the formula (1A) is preferably a compound represented by the following formula (1A′) from the viewpoint of solubility in organic solvents.

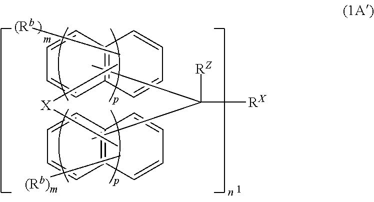

(wherein, R^{b}, X, m and p are the same as defined in the formula (1A); R^{x} represents an n-valent group having 1 to 40 carbon atoms or a single bond; R^{z} represents a hydrogen atom, an alkyl group having 1 to 30 carbon atoms or an aryl group having 6 to 30 carbon atoms; and n¹ is an integer of 1 to 4.)

In the formula (1A′), n¹ “>C(R^{z})—” and one R^{x} correspond to a 2 n-valent group R^{a} as a whole. Herein, when n¹=1, R^{x} represents an n-valent group having 1 to 40 carbon atoms; and when n¹=2 to 4, R^{x} represents an n-valent group having 1 to 40 carbon atoms or a single bond.

The compound represented by the formula (1A′) is preferably a compound represented by the following formula (1′) from the viewpoint of ease of production.

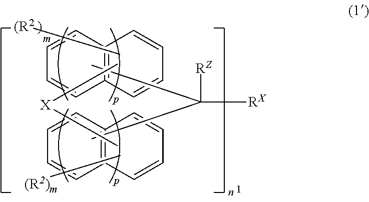

(wherein, R^{x}, R^{z}, X, m, n¹ and p are the same as defined in the formula (1A′); and R² is the same as defined in the formula (1).)

The compound represented by the formula (1′) is preferably a compound represented by the following formula (1-1′) from the viewpoint of heat resistance.

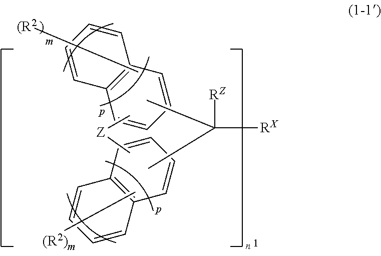

(wherein, R^{x}, R^{z}, R², m, n¹ and p are the same as defined in the formula (1′); and Z is the same as defined in the formula (1-1).)

In addition, the compound represented by the formula (1-1′) is preferably a compound represented by the following formula (1-2′) from the viewpoint of supply of raw materials.

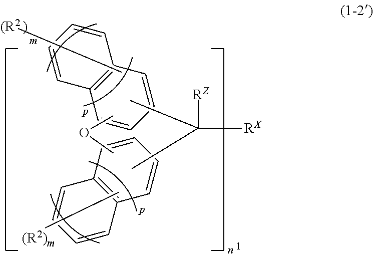

(wherein R^{x}, R^{z}, R², m, n¹ and p are the same as defined in the formula (1-1′).)

Furthermore, the compound represented by the formula (1-2′) is preferably a compound represented by the following formula (1-3′) from the viewpoint of thermosetting properties and dissolution stability.

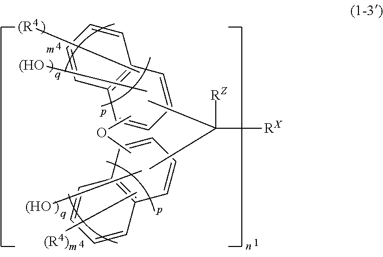

(wherein, R^{x}, R^{z}, n¹ and p are the same as defined in the formula (1-2′); and R⁴, m⁴ and q are the same as defined in the formula (1-3).)

Furthermore, the compound represented by the formula (1-3′) is preferably a compound represented by the following formula (1-4′) from the viewpoint of heat resistance and dissolution stability.

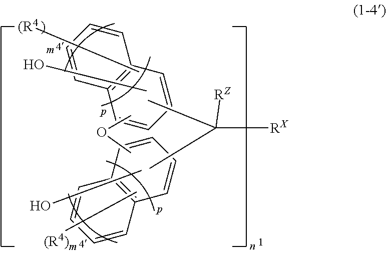

(wherein, R^{x}, R^{z}, R⁴, n¹ and p are the same as defined in the formula (1-2′); and m^{4′} is the same as defined in the formula (1-4).)

Furthermore, the compound represented by the formula (1A′) is preferably a compound represented by the following formula (3′) from the viewpoint of heat resistance and dissolution stability.

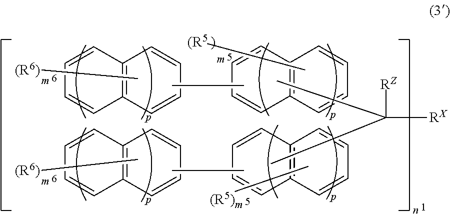

(wherein, R^{x}, R^{z}, n¹ and p are the same as defined in the formula (1A′); and R⁵, R⁶, m⁵ and m⁶ are the same as defined in the formula (3).)

Furthermore, the compound represented by the formula (3′) is preferably a compound represented by the following formula (3-1′) from the viewpoint of heat resistance and dissolution stability.

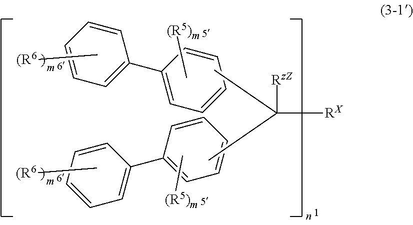

(wherein, R^{x}, R^{z}, R⁵, R⁶, n¹ and p are the same as defined in the formula (1A′); and m^{5′} and m^{6′} are the same as defined in the formula (3-1).)

In the present embodiment, R^{x} preferably represents an aryl group having 7 or more carbon atoms; and R^{z} preferably represents a hydrogen atom or a methyl group. Examples of the aryl group having 7 or more carbon atoms include, but not limited to, a biphenyl group, a naphthalene group, an anthracene group and a pyrene group.

Specific examples of the compound represented by the formula (1) are illustrated below without limitation.

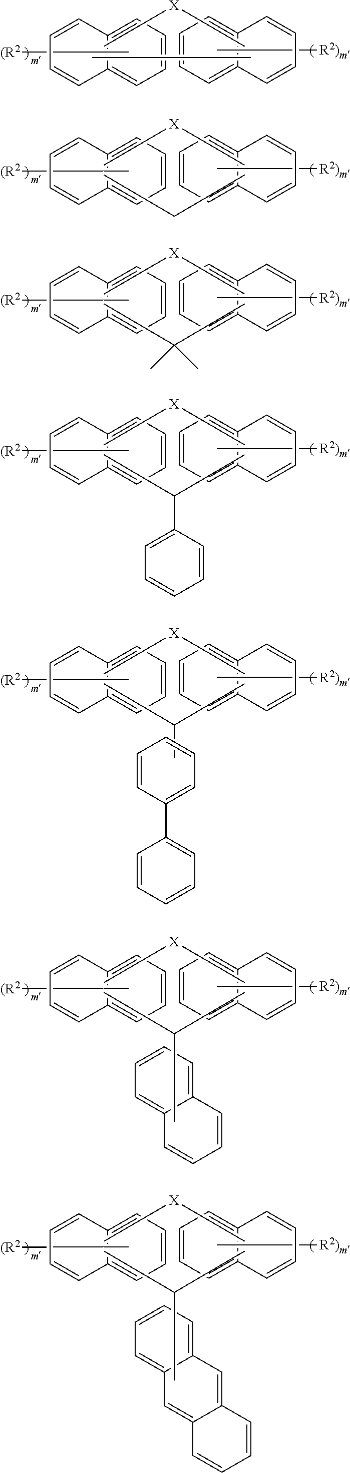

In the above formulae, R² and X are the same as defined in the formula (1). m′ is an integer of 0 to 7. Herein, at least one R² represents one selected from a hydroxyl group and a thiol group, and all of m′ cannot be 0 at the same time.

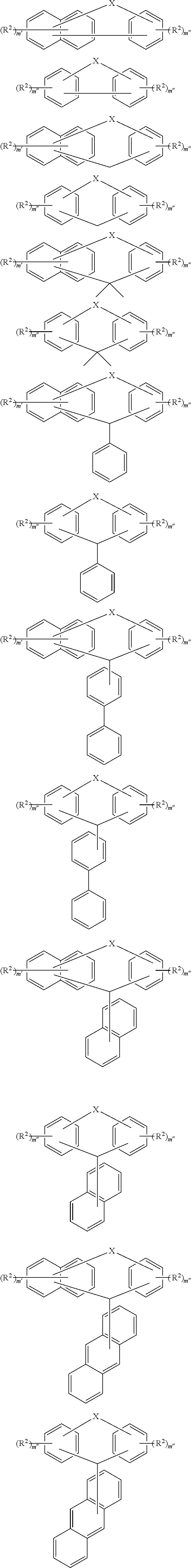

In the above formulae, R² and X are the same as defined in the formula (1).

m′ is an integer of 0 to 7. m″ is an integer of 0 to 5. Herein, at least one R² represents one selected from a hydroxyl group and a thiol group, and all of m′ and m″ cannot be 0 at the same time.

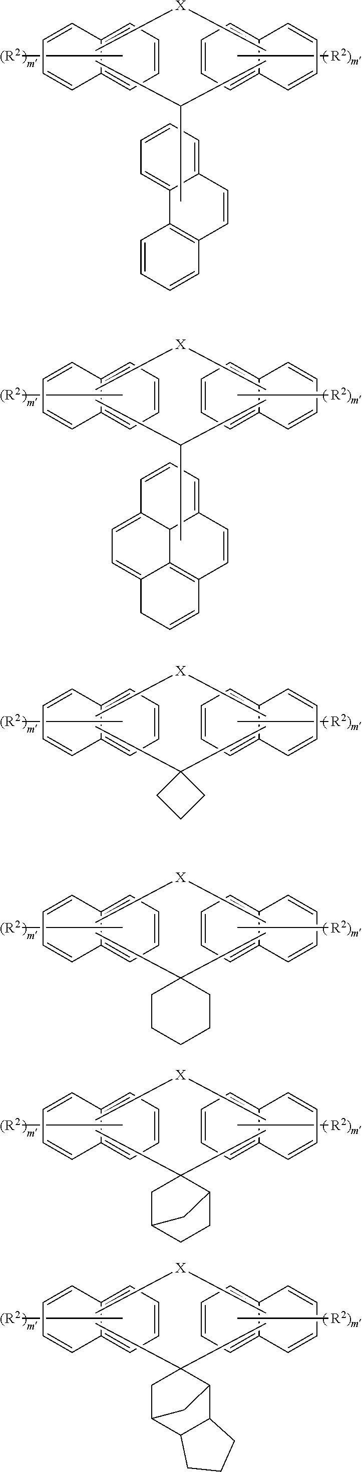

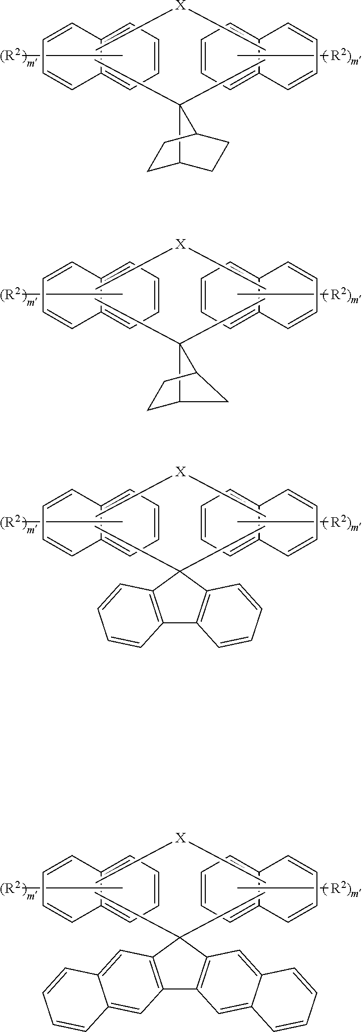

In the above formulae, R², X and m′ are the same as defined above. Herein, at least one R² represents one selected from a hydroxyl group and a thiol group, and all m′ cannot be 0 at the same time.

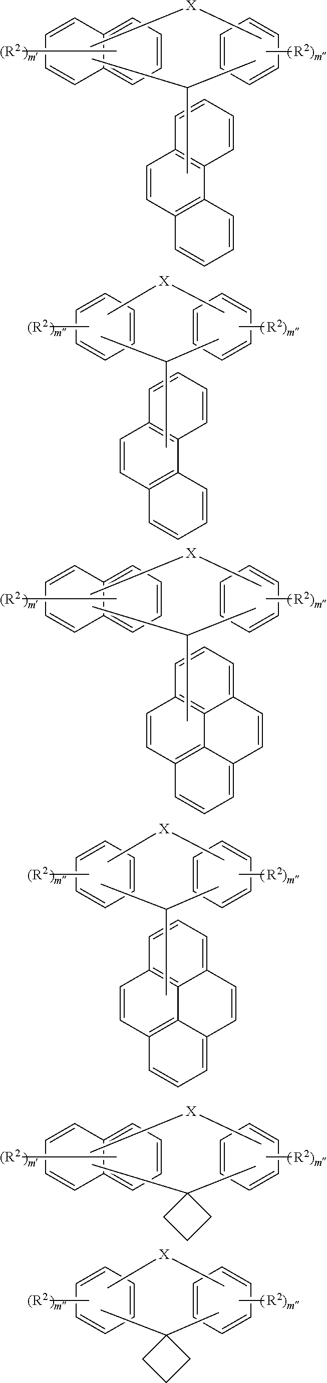

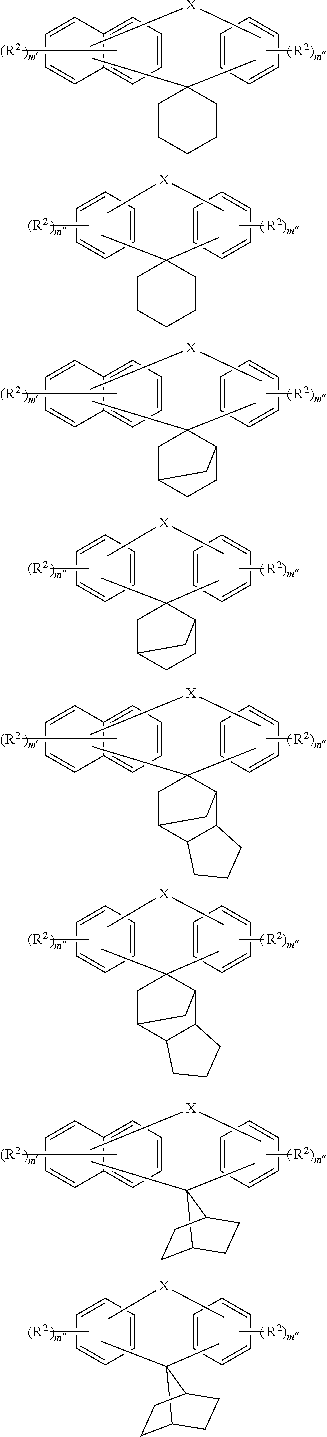

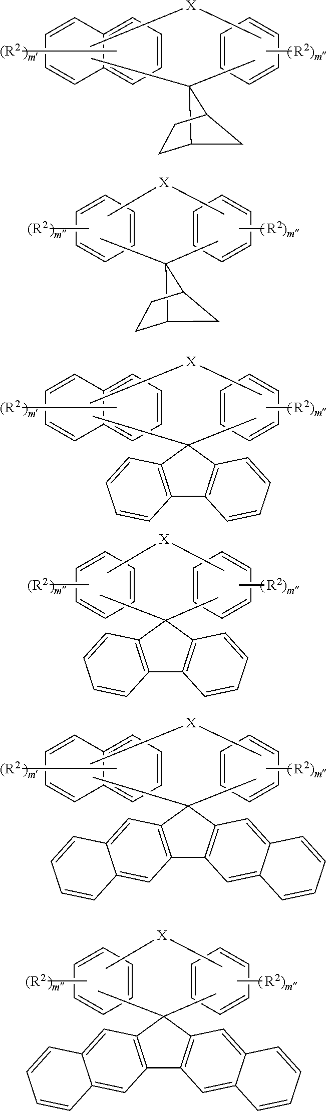

In the above formulae, R² and X are the same as defined in the formula (1). m′ is an integer of 0 to 7. m″ is an integer of 0 to 5. Herein, at least one R² represents one selected from a hydroxyl group and a thiol group, and all of m′ and m″ cannot be 0 at the same time.

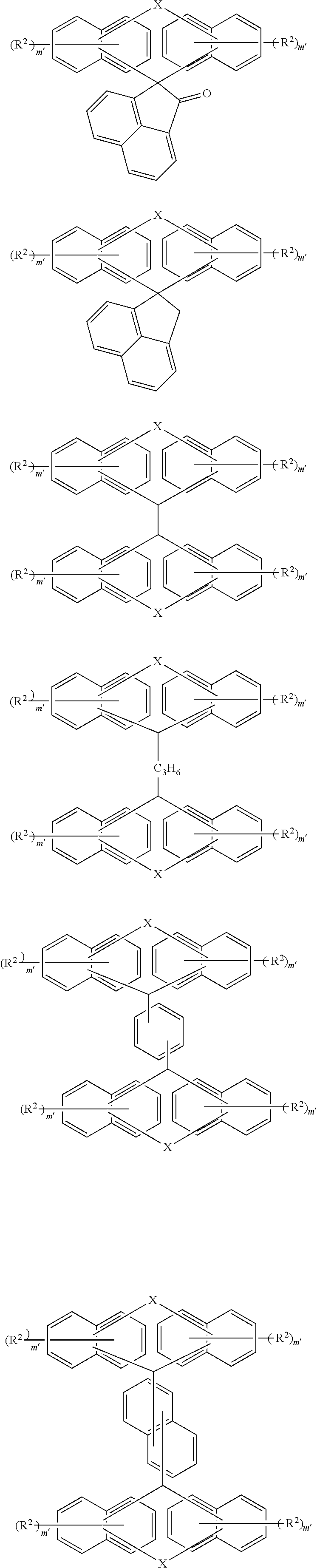

In the above formulae, R² and X are the same as defined in the formula (1). m′ is an integer of 0 to 7. Herein, at least one R² represents one selected from a hydroxyl group and a thiol group, and all m′ cannot be 0 at the same time.

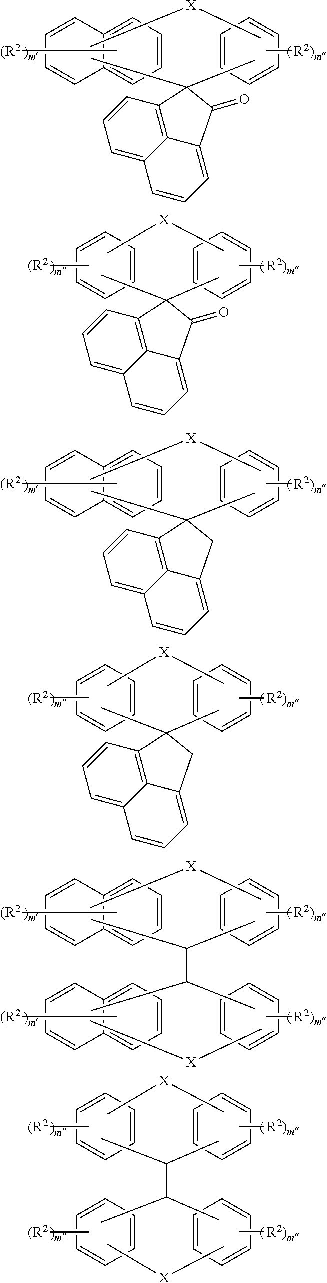

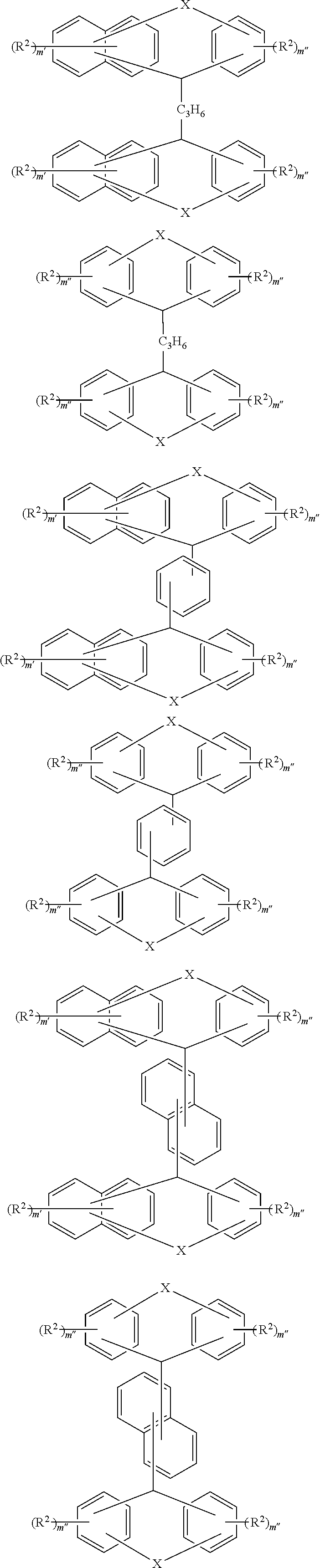

In the above formulae, R² and X are the same as defined in the formula (1). m′ is an integer of 0 to 7. m″ is an integer of 0 to 5. Herein, at least one R² represents one selected from a hydroxyl group and a thiol group, and all m′ and m″ cannot be 0 at the same time.

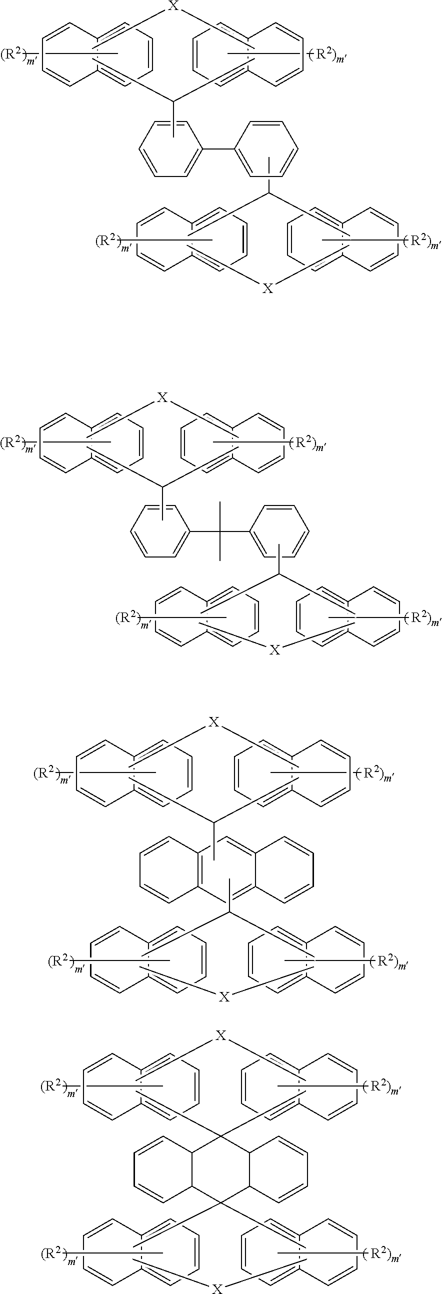

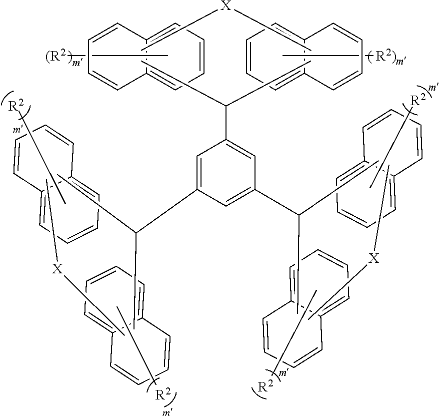

In the above formulae, R² and X are the same as defined in the formula (1). m′ is an integer of 0 to 7. Herein, at least one R² represents one selected from a hydroxyl group and a thiol group, and all m′ cannot be 0 at the same time.

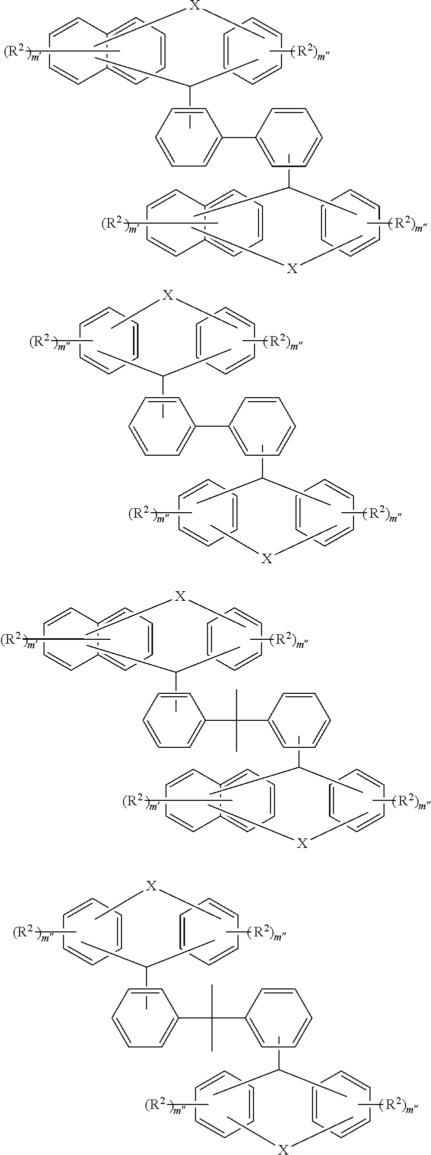

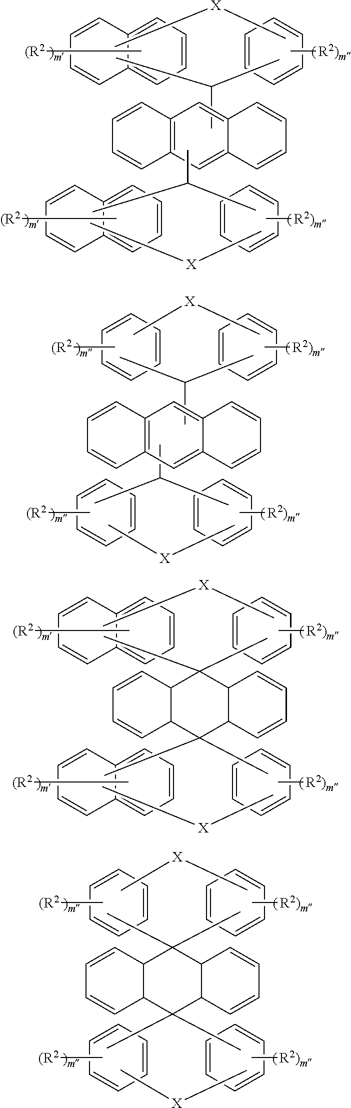

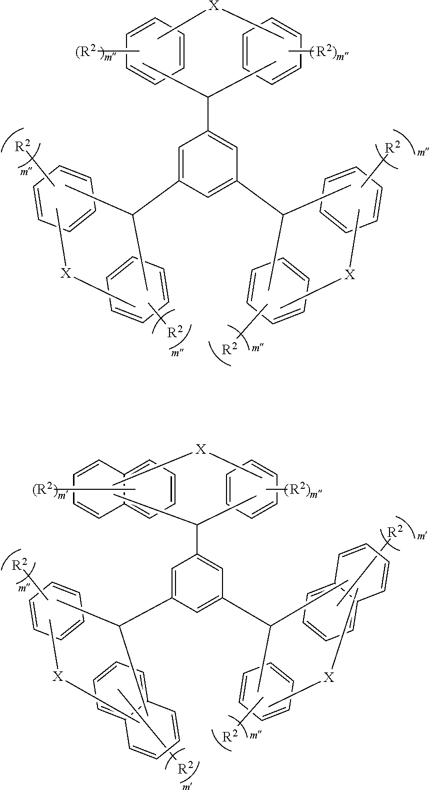

In the above formulae, R² and X are the same as defined in the formula (1). m′ is an integer of 0 to 7. m″ is an integer of 0 to 5. Herein, at least one R² represents one selected from a hydroxyl group and a thiol group, and all of m′ and m″ cannot be 0 at the same time.

Specific examples of the compound represented by the formula (3) are illustrated below without limitation.

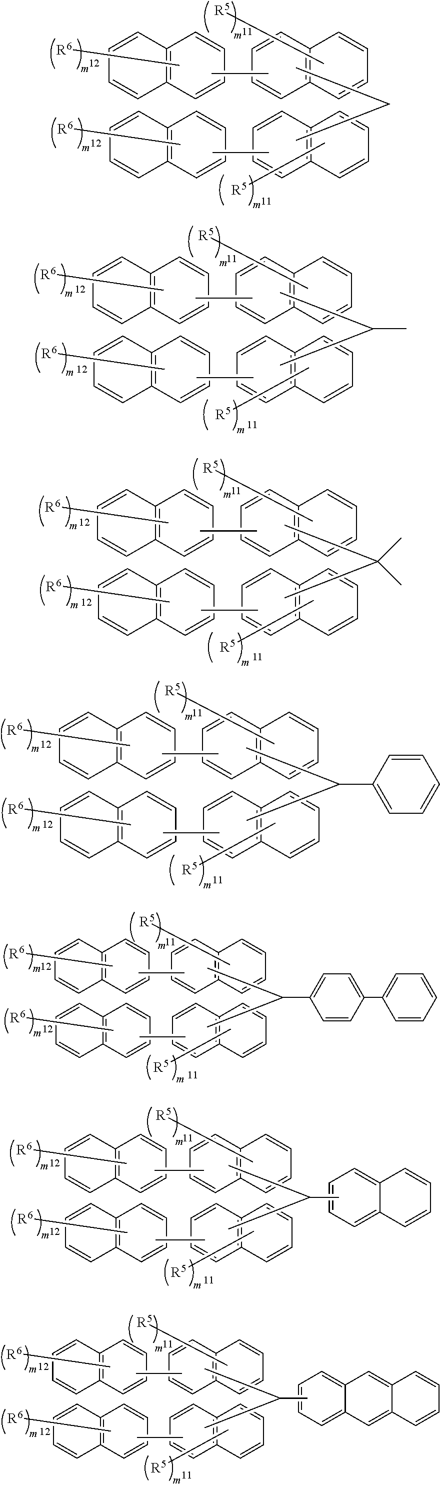

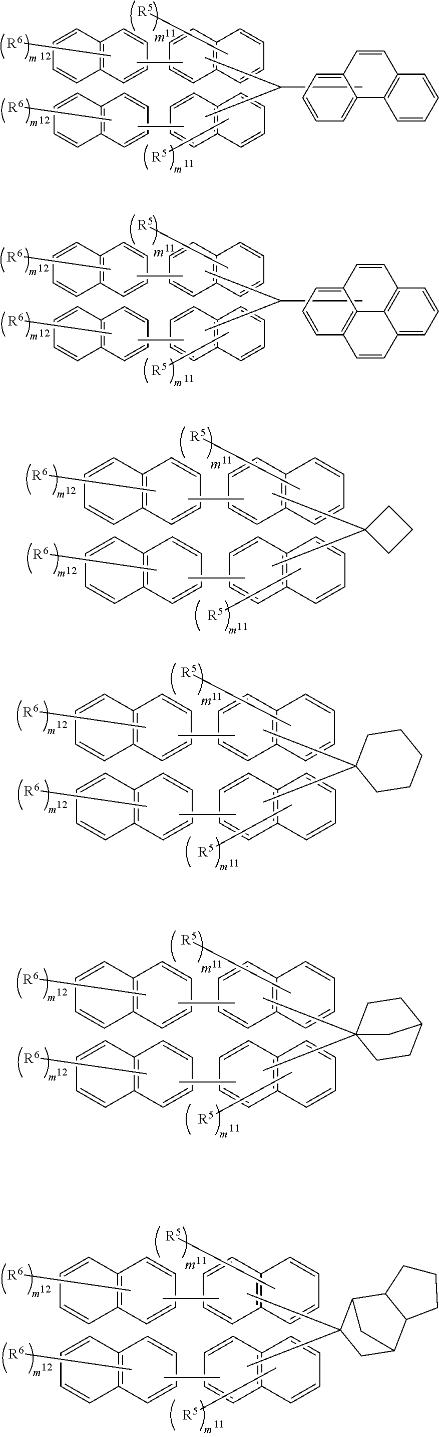

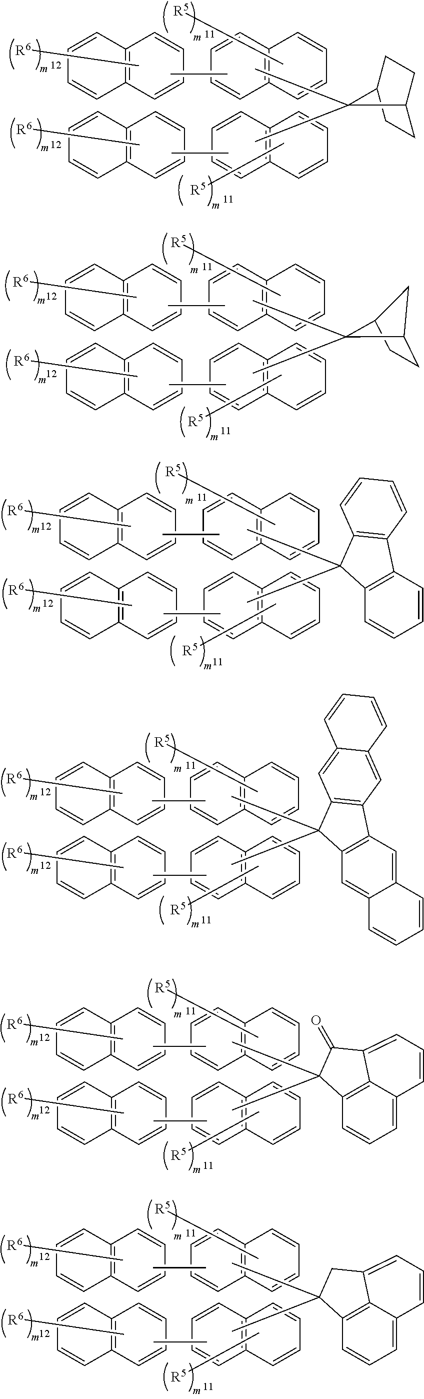

In the above compounds, R⁵ and R⁶ are the same as defined in the formula (3).

m¹¹ is an integer of 0 to 6; and m¹² is an integer of 0 to 7.

Herein, at least one selected from R⁵ and R⁶ represents one selected from a hydroxyl group and a thiol group, and all of m¹¹ and m¹² cannot be 0 at the same time.

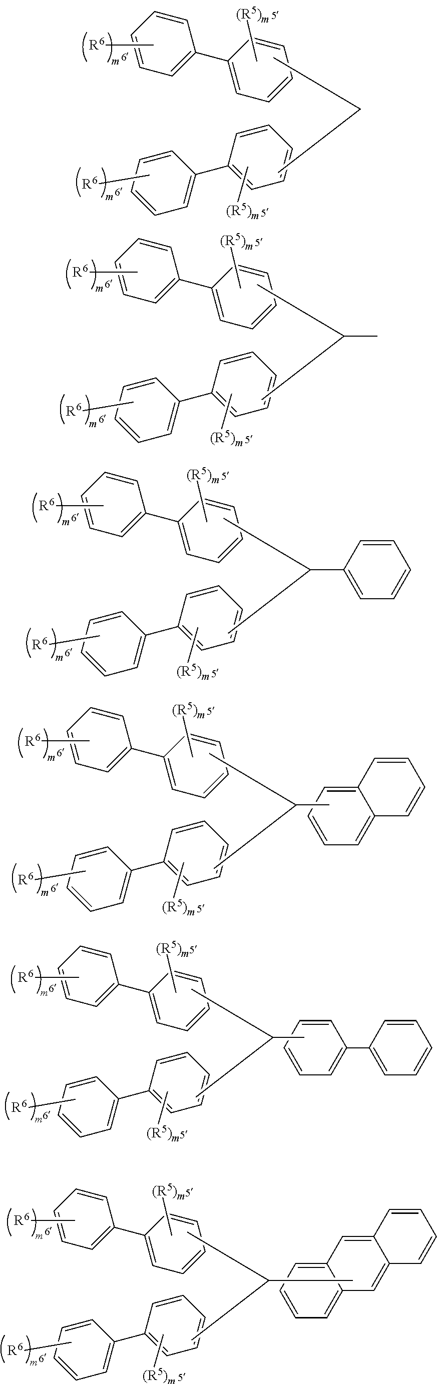

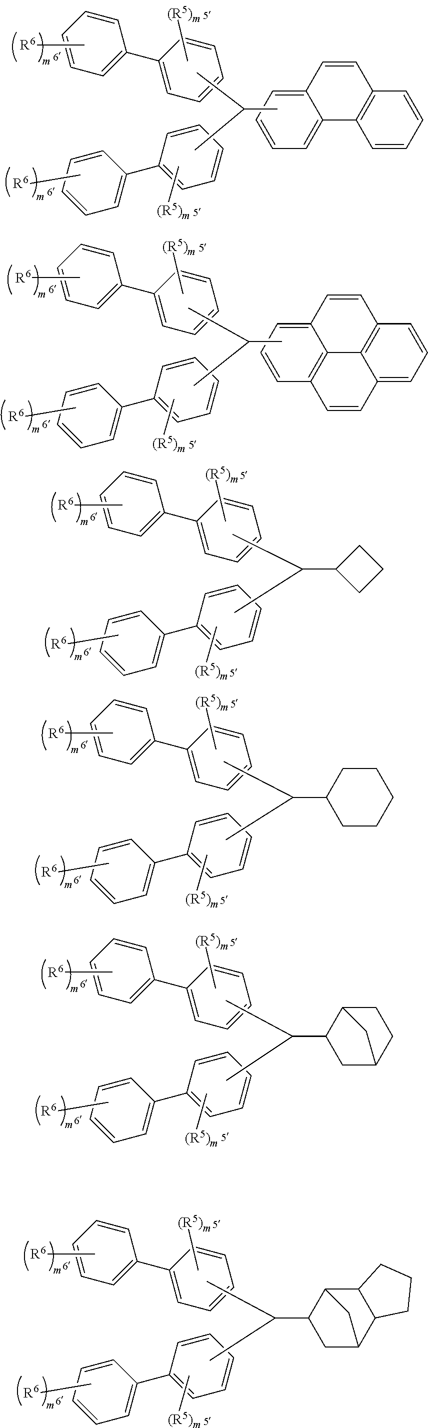

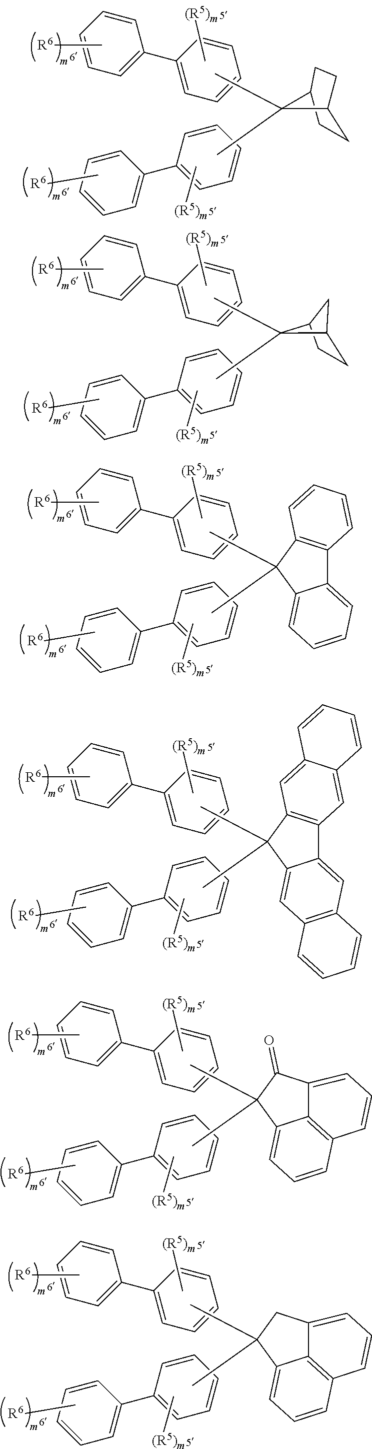

In the above compounds, R⁵ and R⁶ are the same as defined in the formula (3).

Each m^{5′} is independently an integer of 0 to 4; and each m^{6′} is independently an integer of 0 to 5.

Herein, at least one selected from R⁵ and R⁶ represents one selected from a hydroxyl group and a thiol group, and all of m^{5′} and m^{6′} cannot be 0 at the same time.

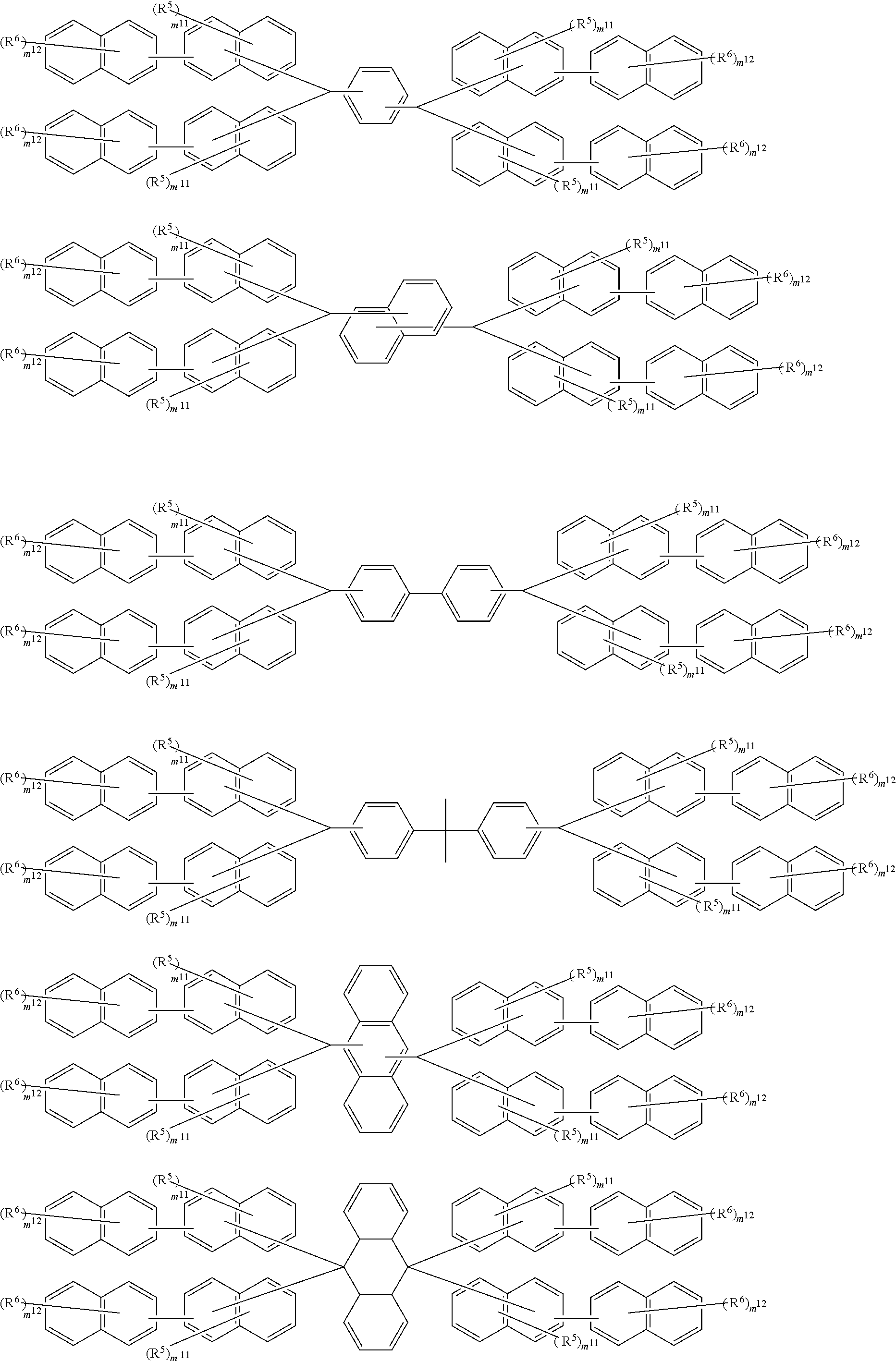

In the above compounds, R⁵ and R⁶ are the same as defined in the formula (3).

m¹¹ is an integer of 0 to 6; and m¹² is an integer of 0 to 7.

Herein, at least one selected from R¹¹ and R¹² represents one selected from a hydroxyl group and a thiol group, and all of m¹¹ and m¹² cannot be 0 at the same time.

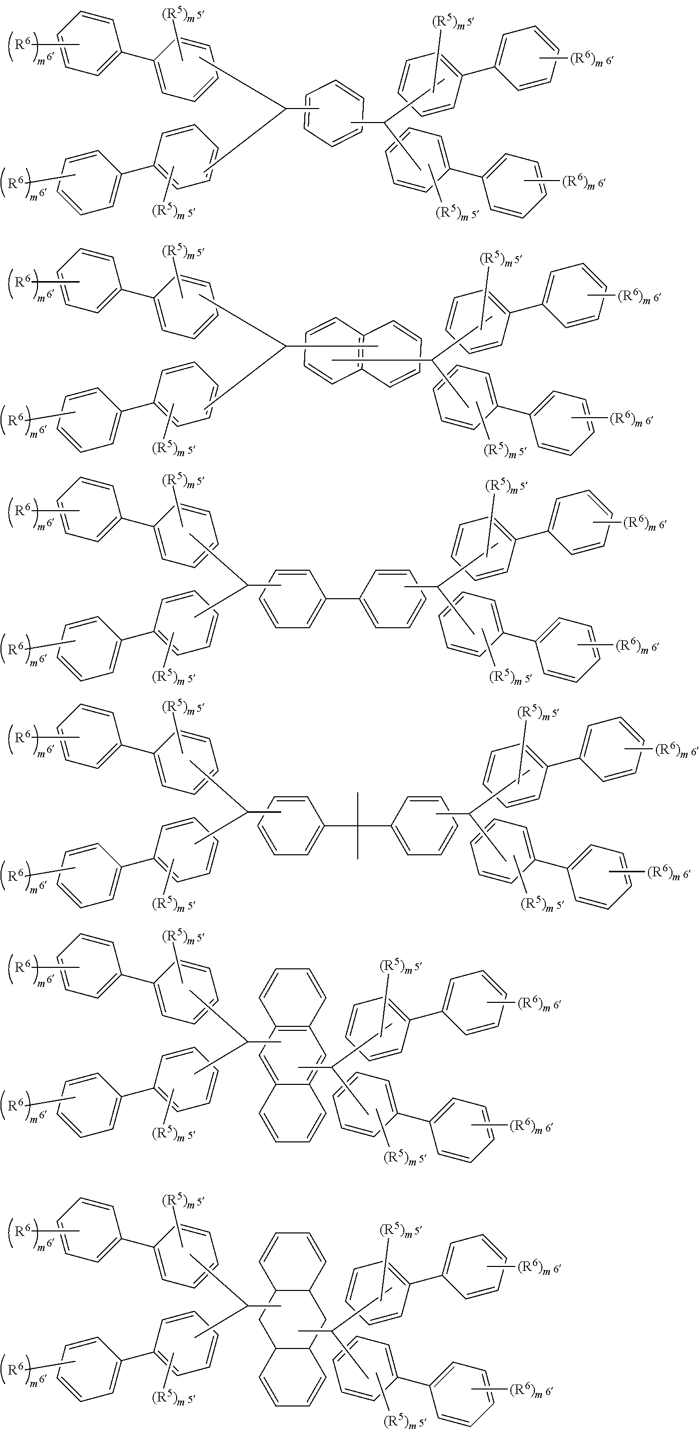

In the above compounds, R⁵ and R⁶ are the same as defined in the formula (1).

Each m^{5′} is independently an integer of 0 to 4; and each m^{6′} is independently an integer of 0 to 5.

Herein, at least one selected from R⁵ and R⁶ represents one selected from a hydroxyl group and a thiol group, and all of m^{5′} and m^{6′} cannot be 0 at the same time.

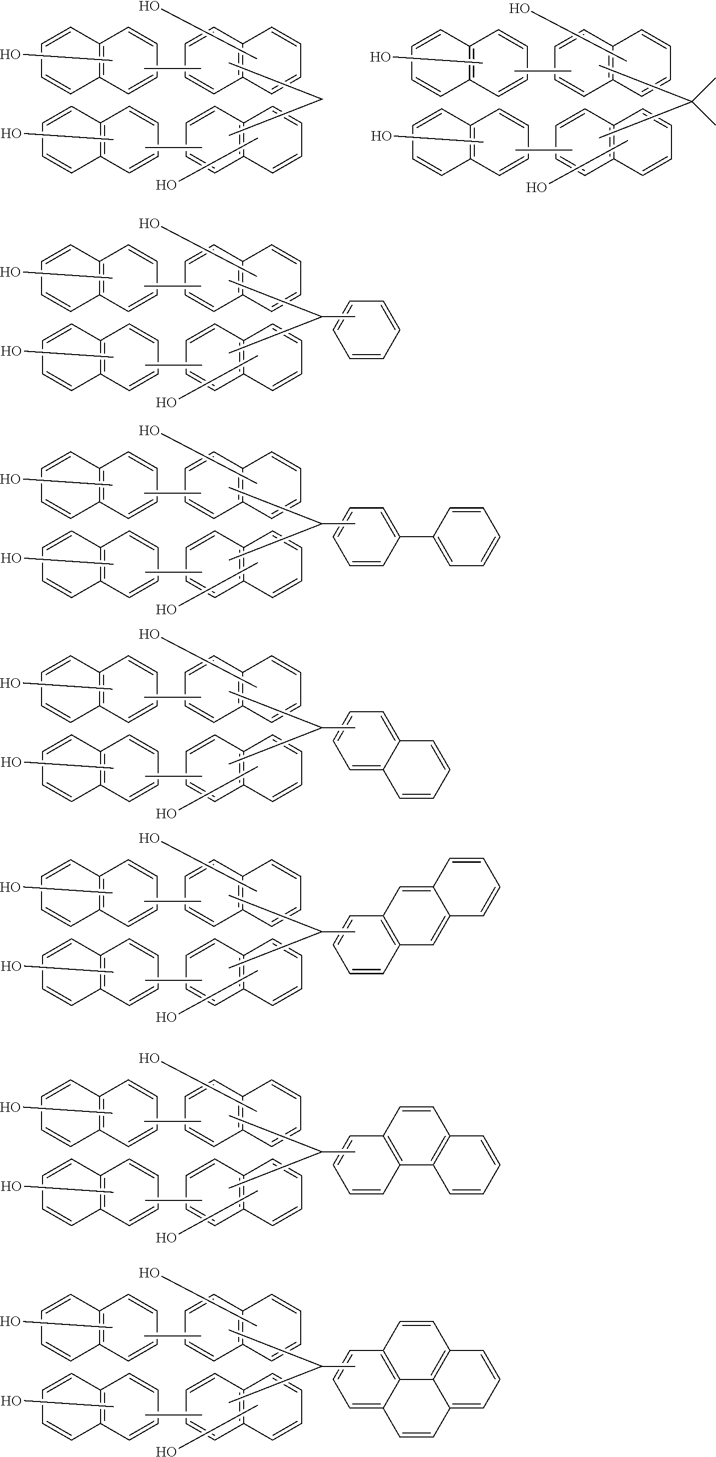

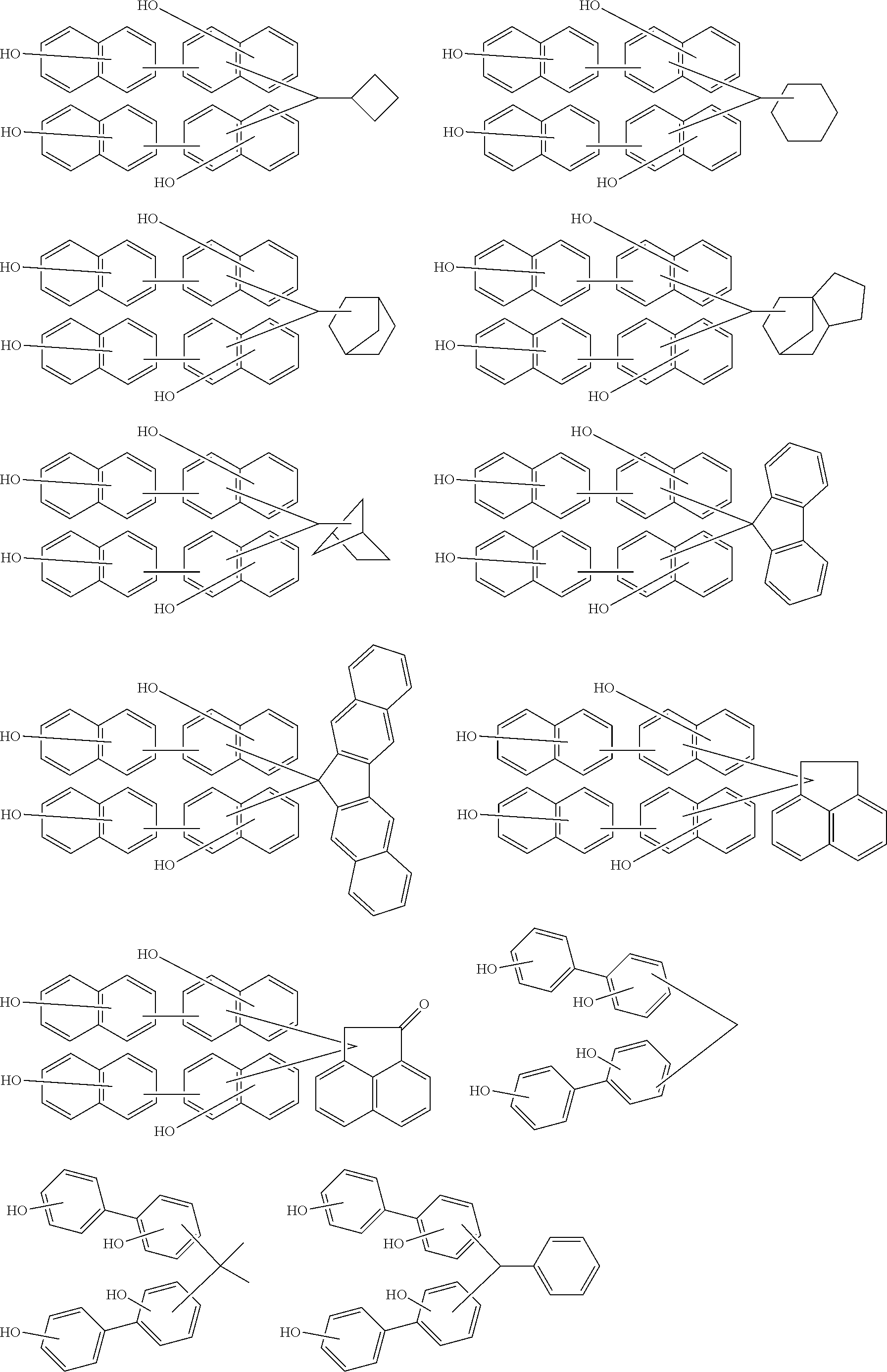

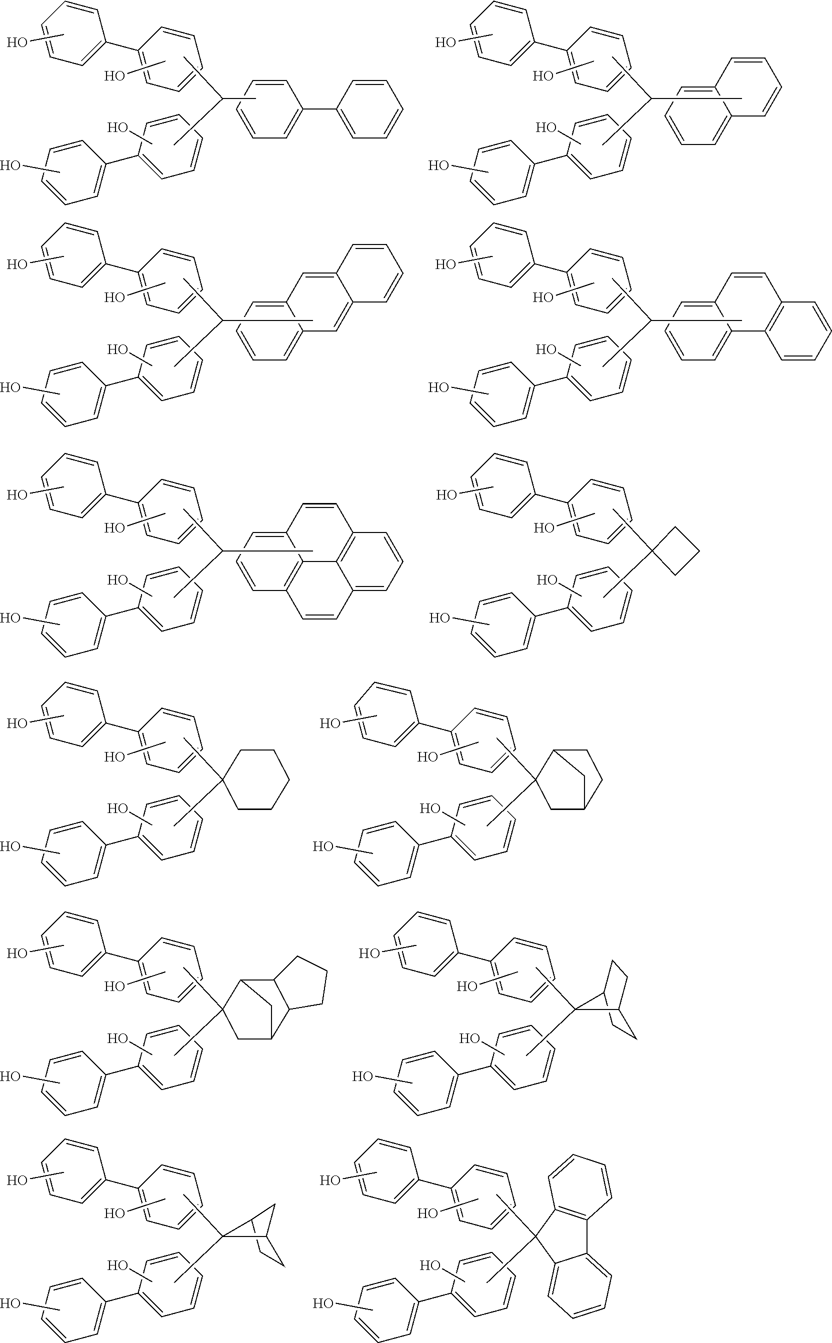

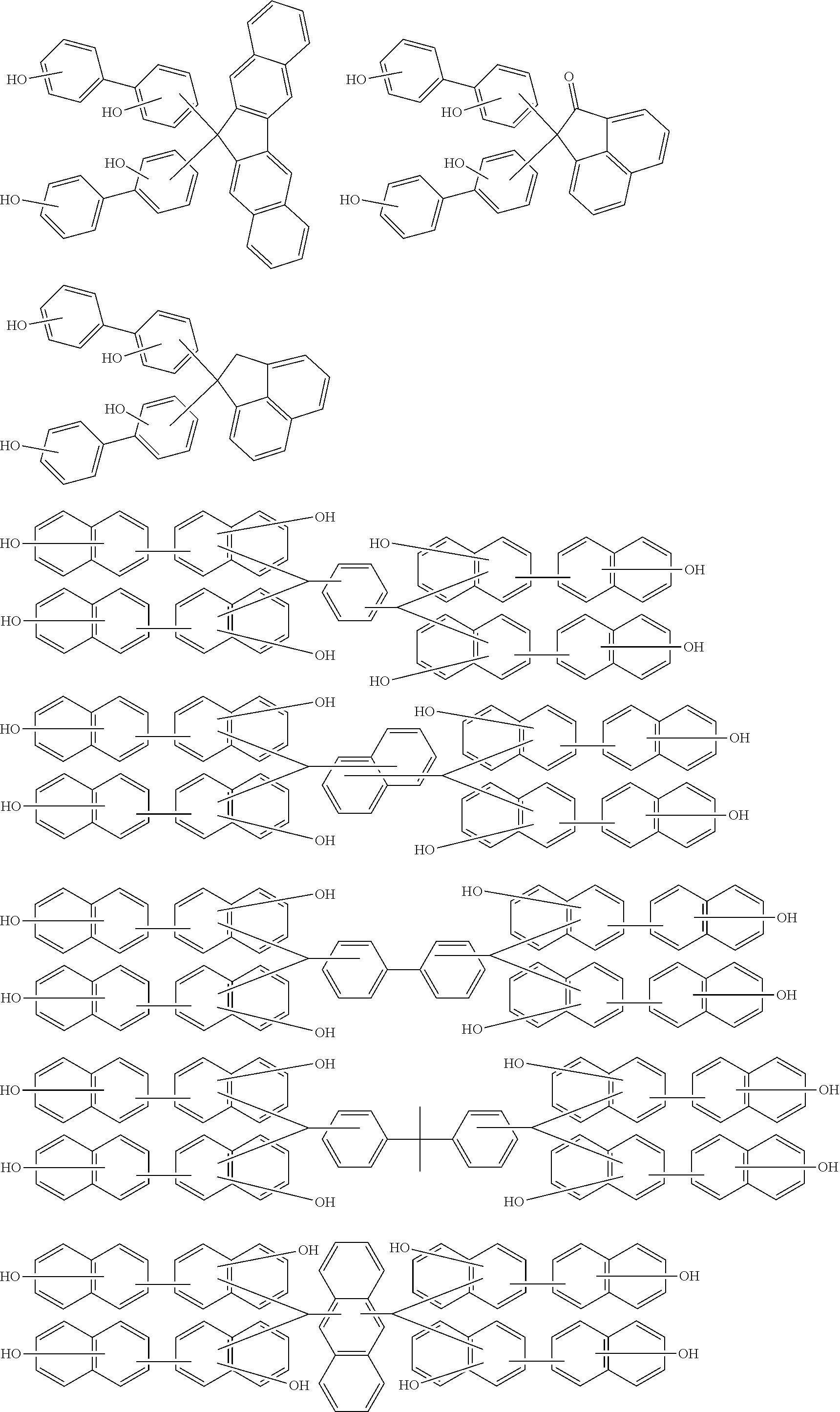

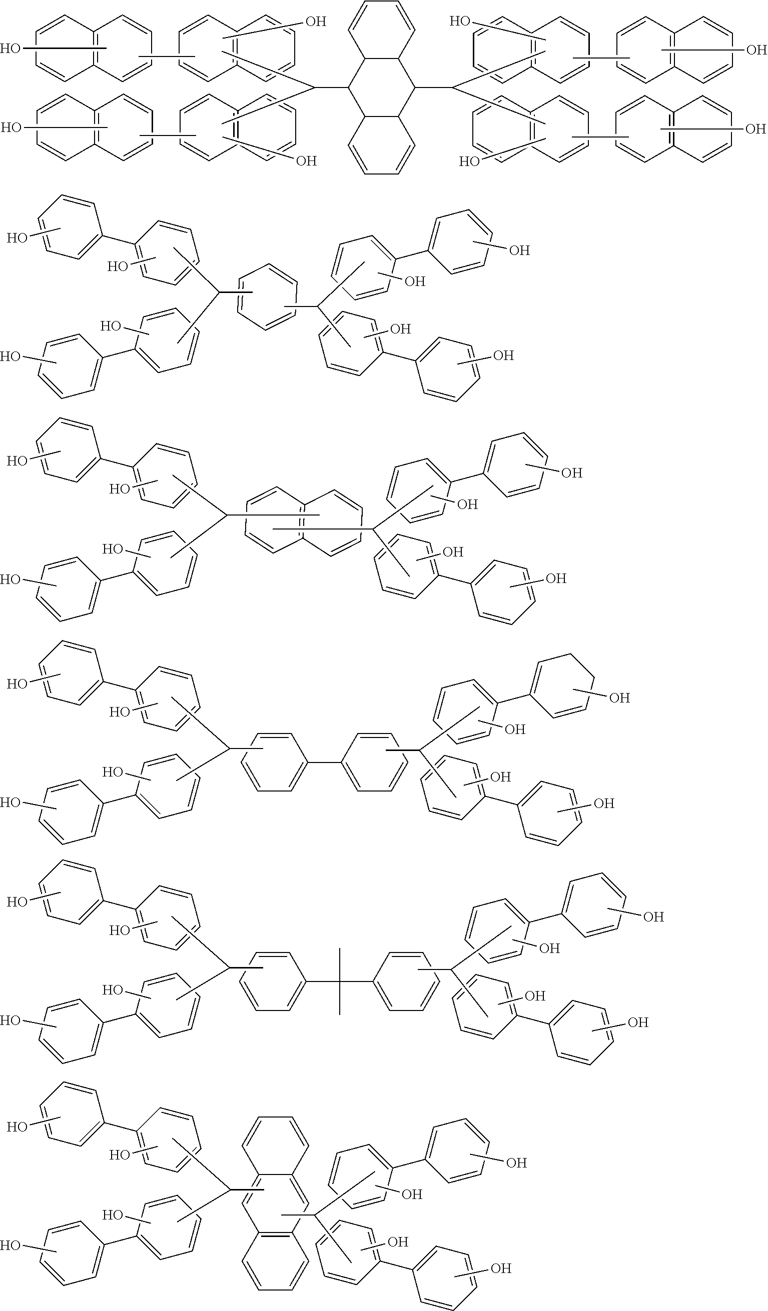

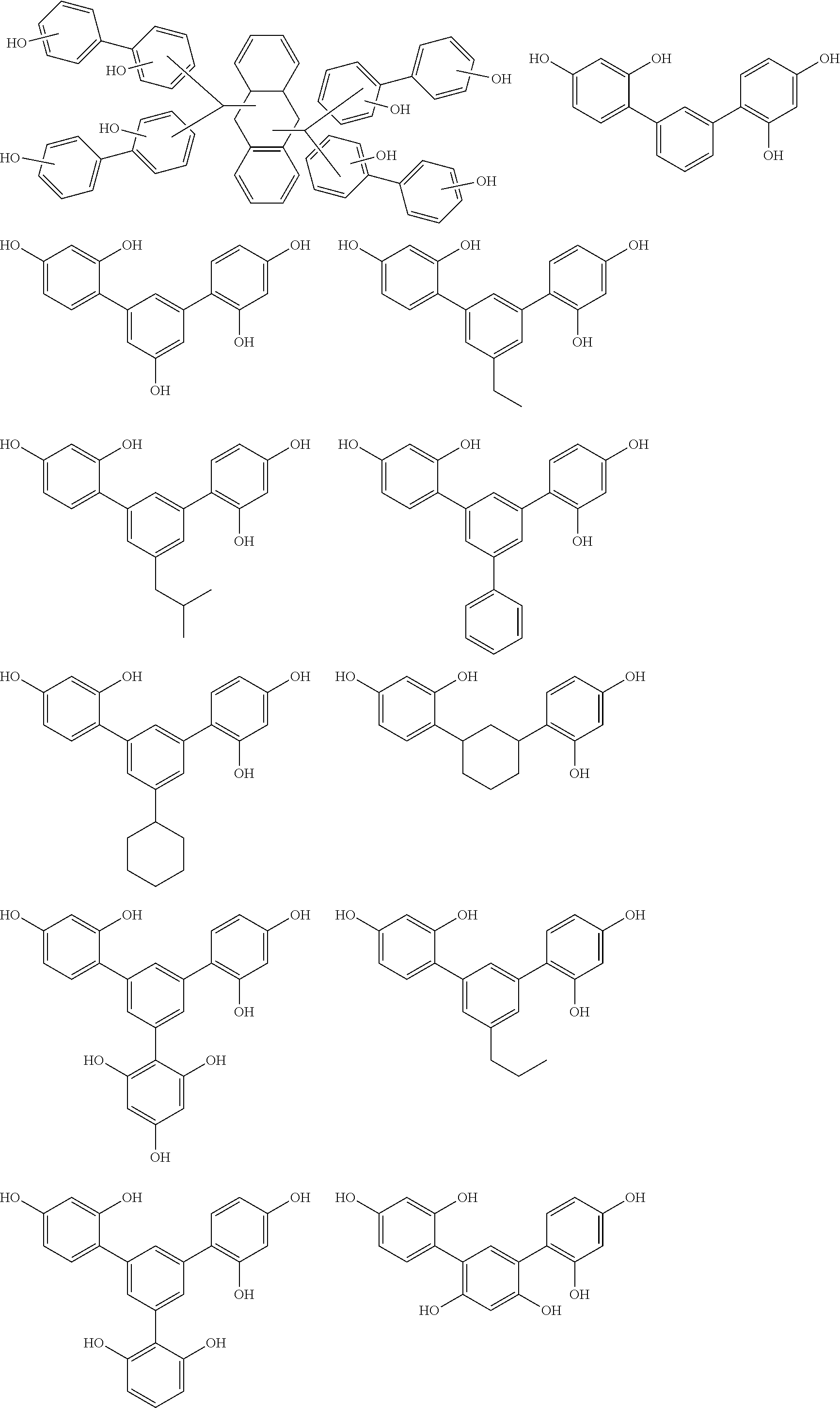

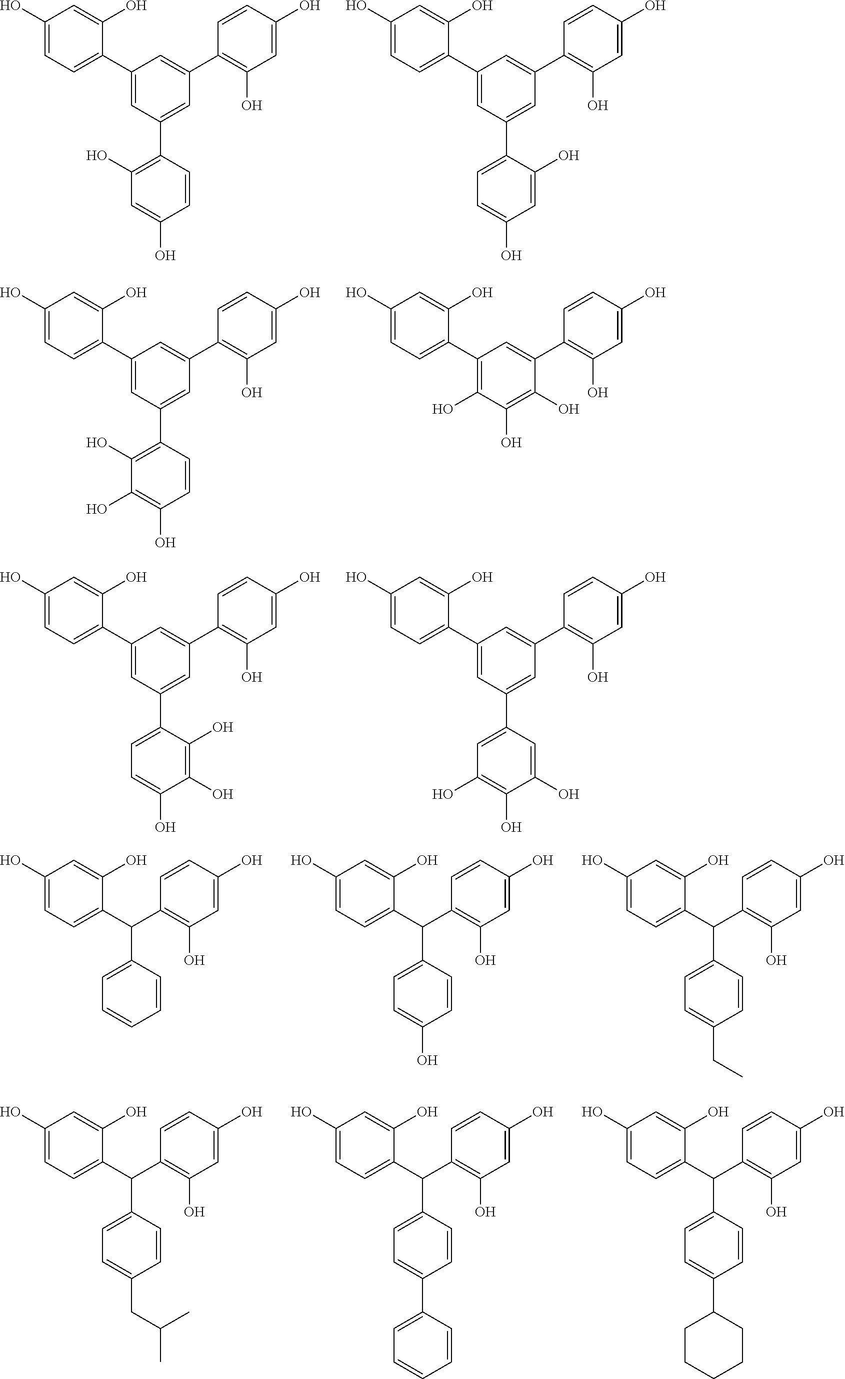

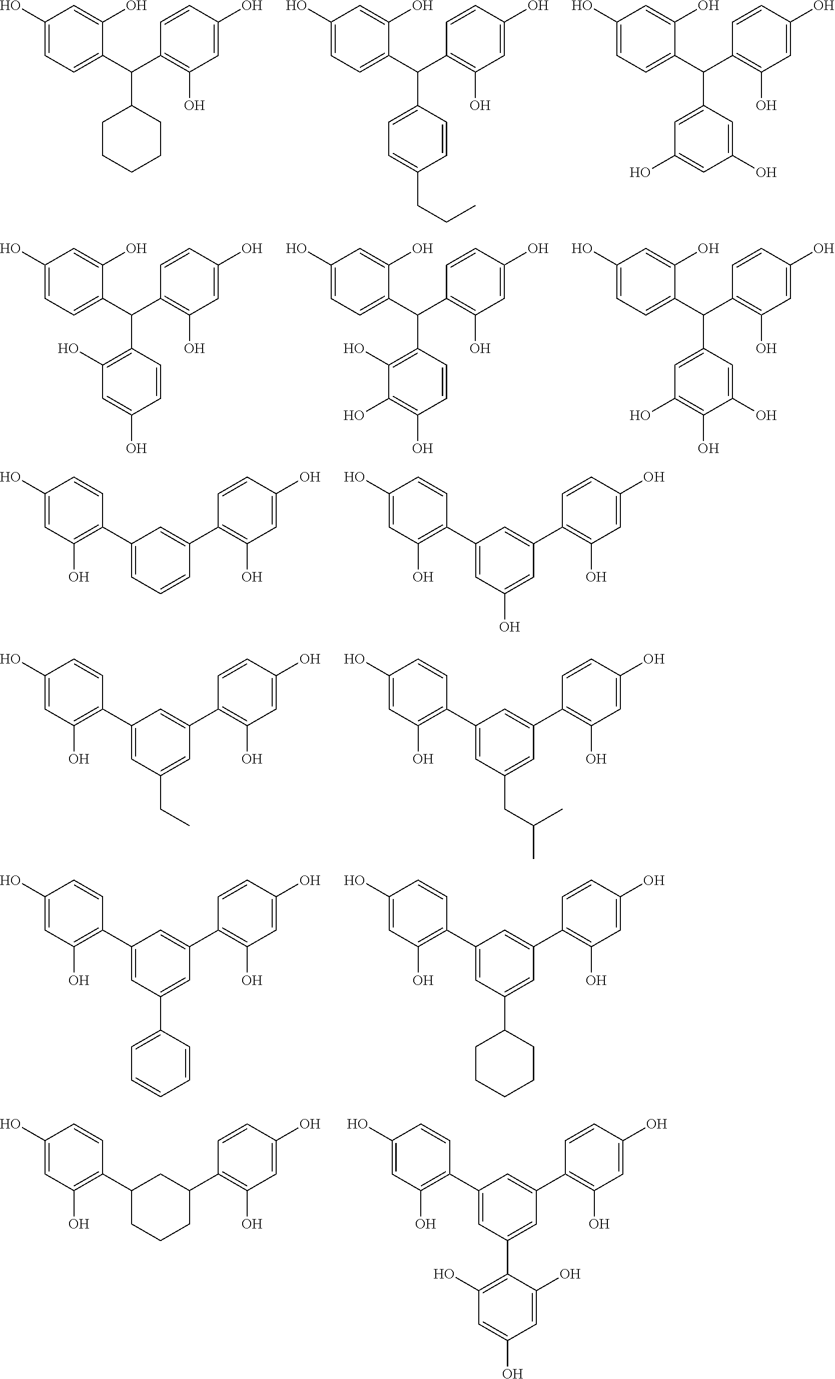

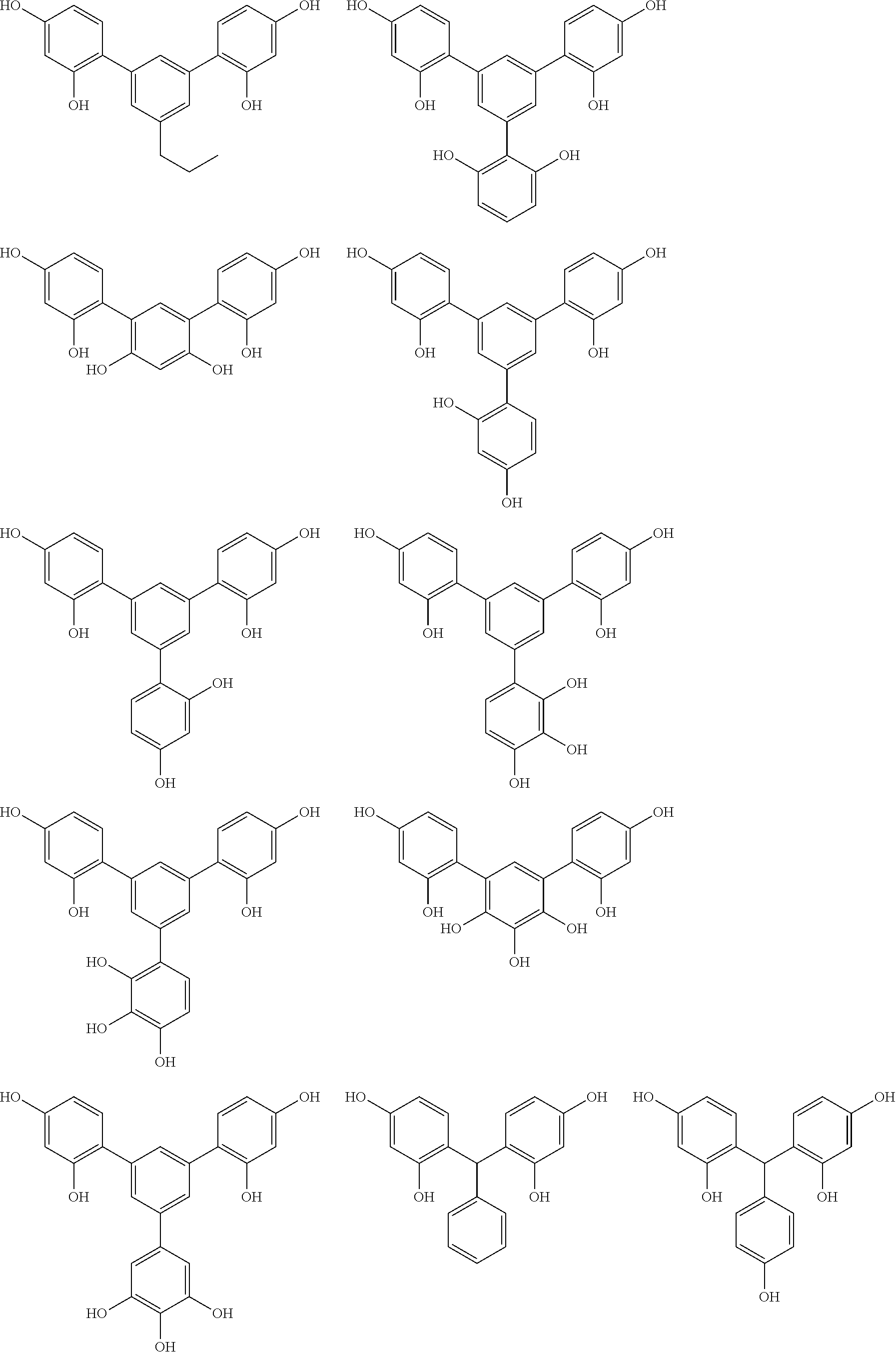

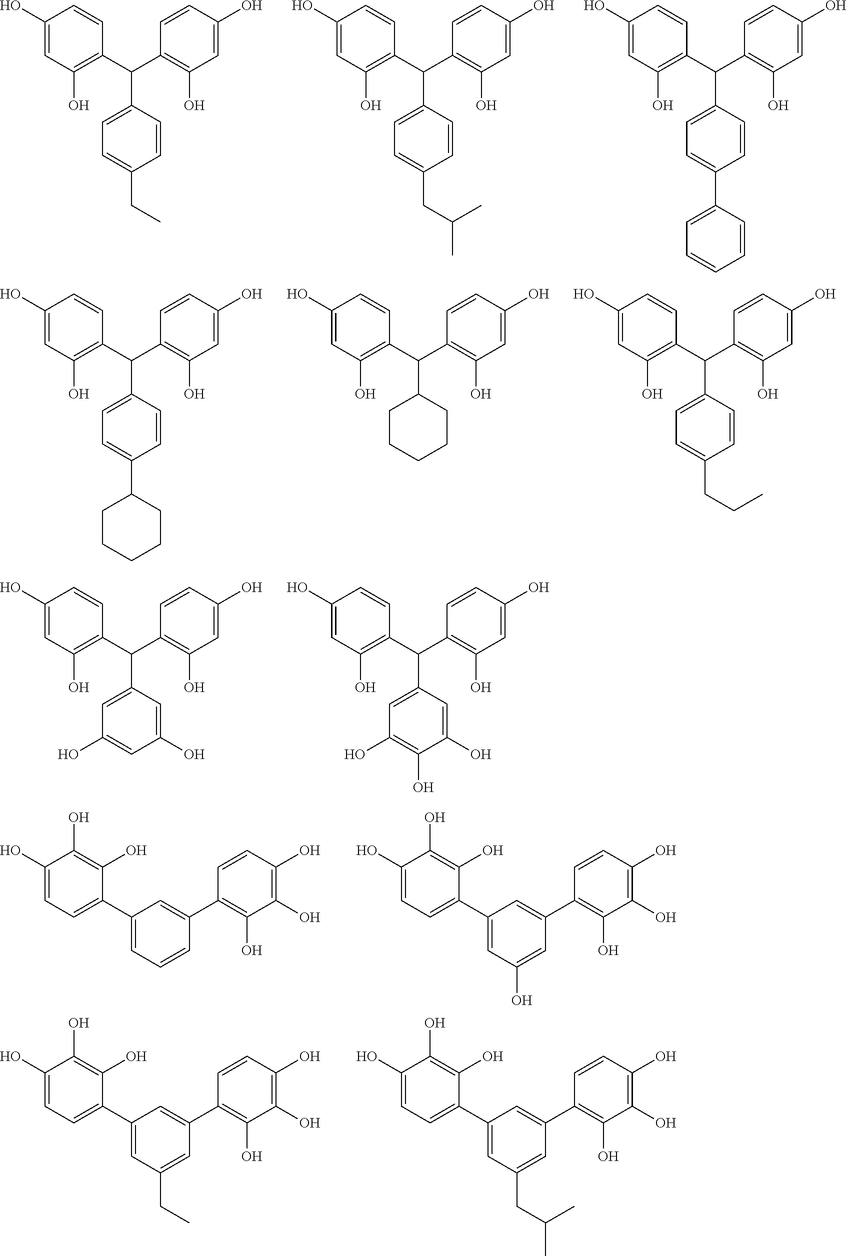

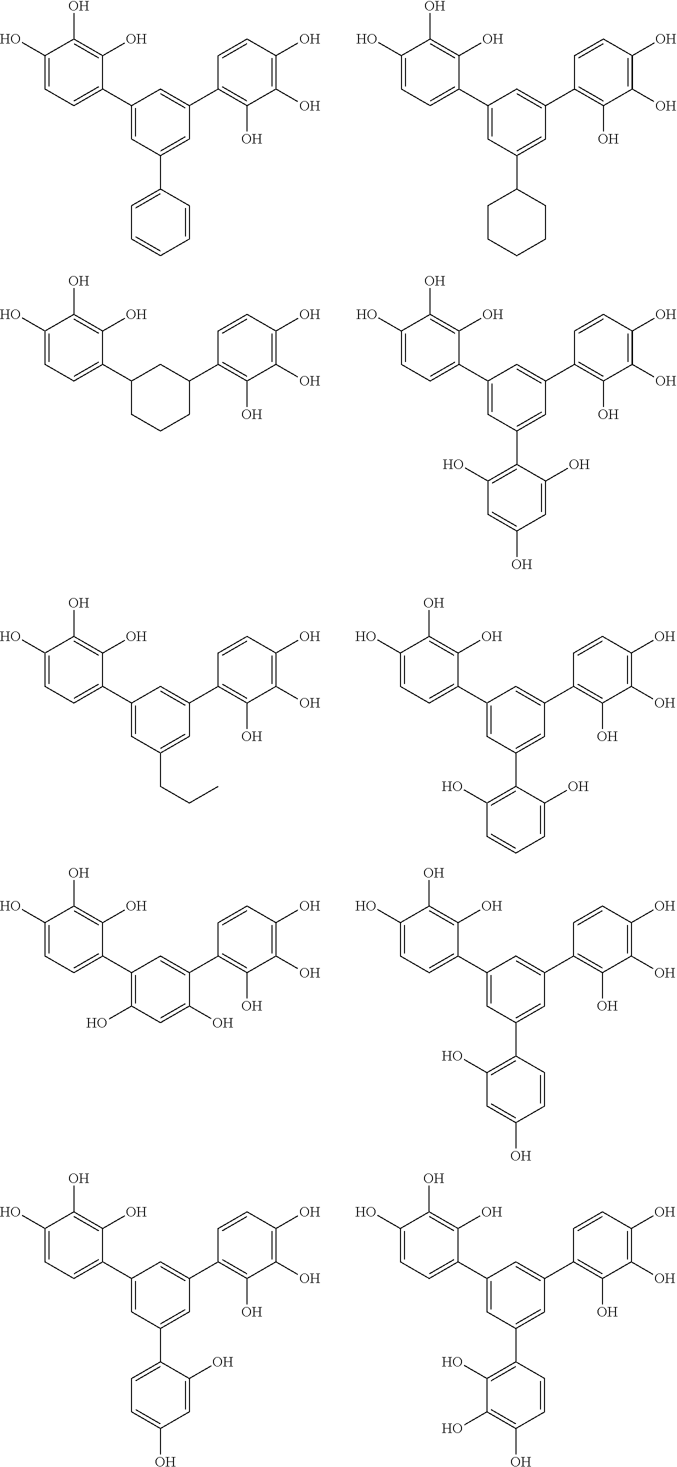

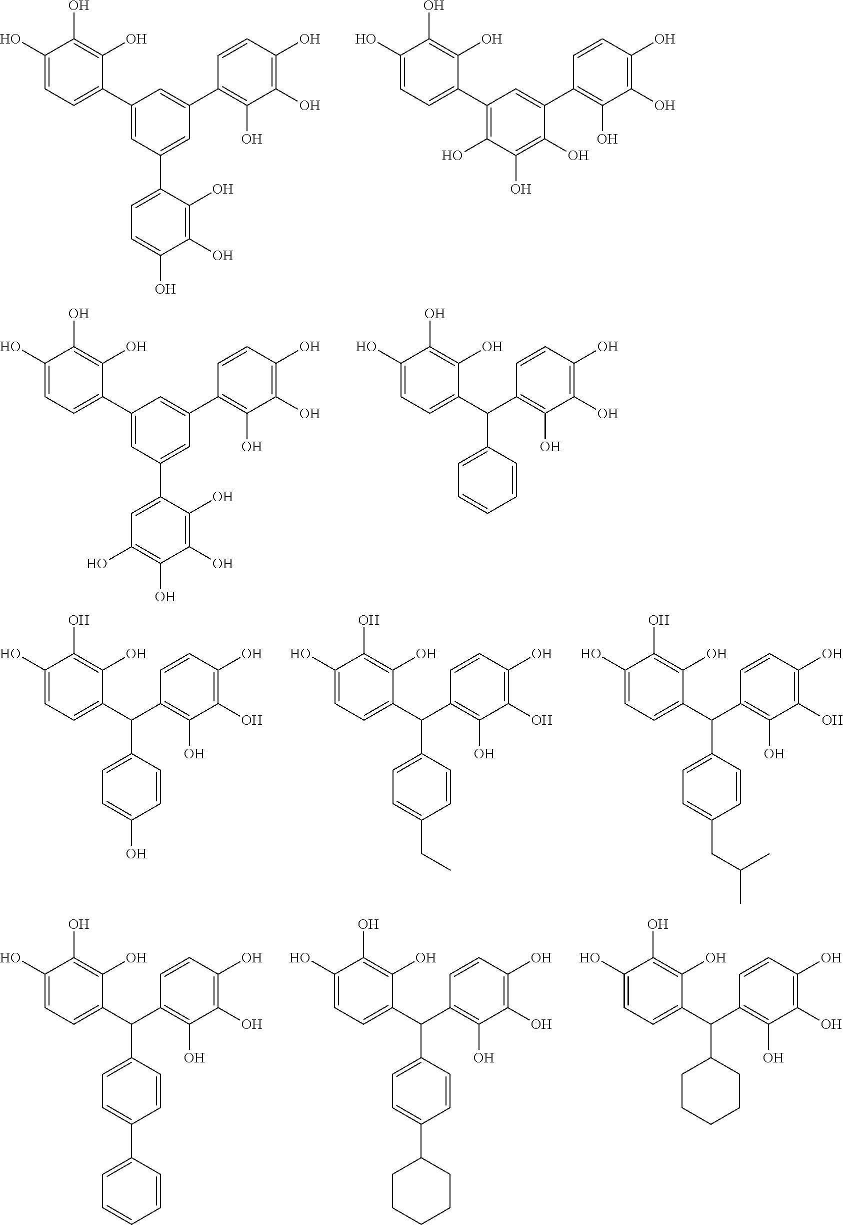

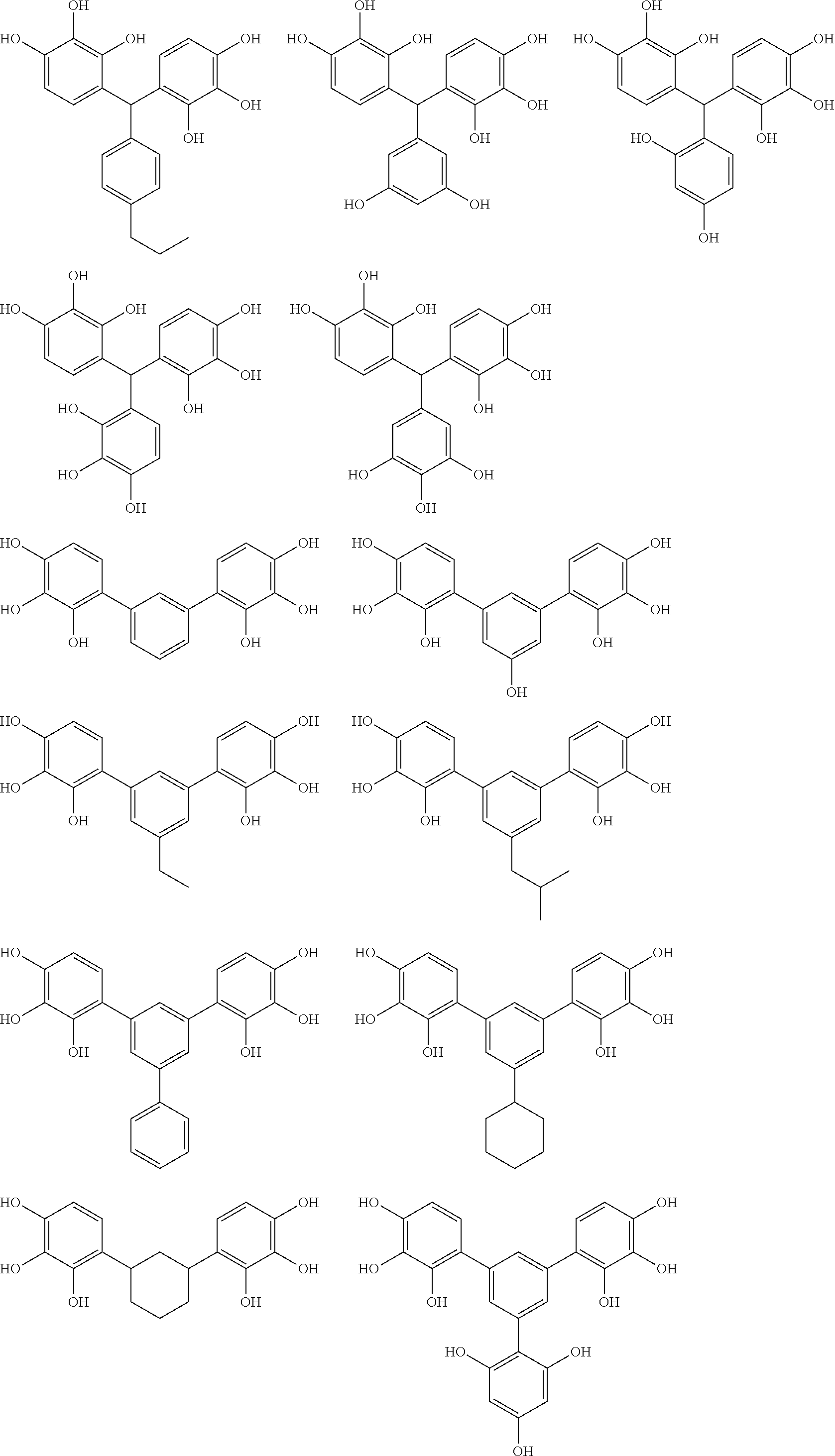

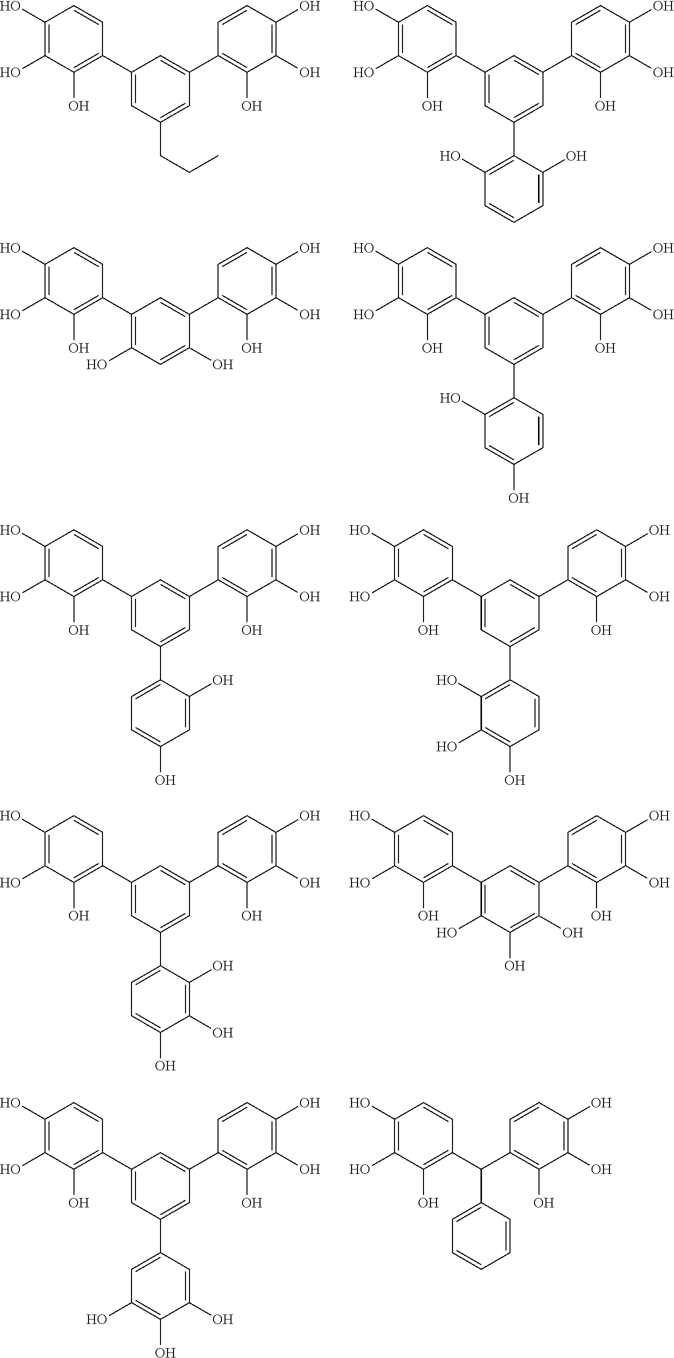

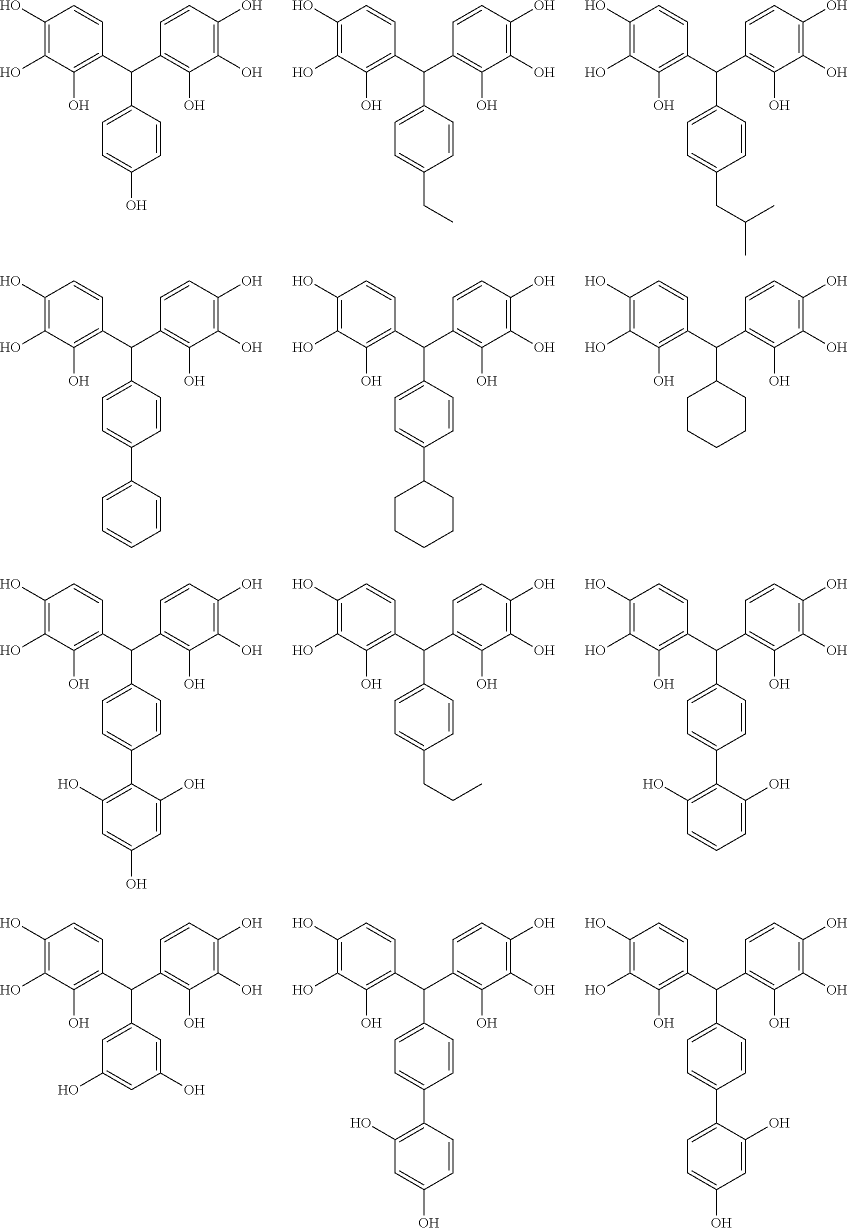

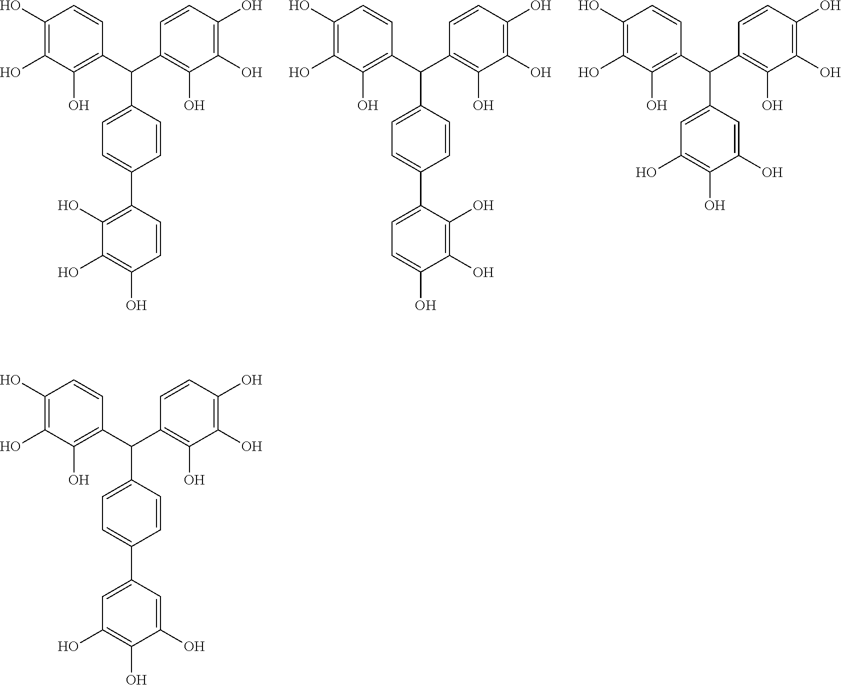 [SMILES: Oc1ccc(-c2ccc(C(c3ccc(O)c(O)c3O)c3ccc(O)c(O)c3O)cc2)c(O)c1O ; Oc1ccc(-c2ccc(C(c3ccc(O)c(O)c3O)c3ccc(O)c(O)c3O)cc2)c(O)c1O ; Oc1cc(C(c2ccc(O)c(O)c2O)c2ccc(O)c(O)c2O)cc(O)c1O ; Oc1cc(-c2ccc(C(c3ccc(O)c(O)c3O)c3ccc(O)c(O)c3O)cc2)cc(O)c1O]

### [Method for Preparing Compound Represented by Formula (1A) and Compound Represented by Formula (1A′)]

A compound represented by the formula (1A) and a compound represented by the formula (1A′), which are used in the present embodiment, can be appropriately synthesized by applying a known method without particular limitation. These compounds can be produced, for example, by the methods described in International Publication No. WO2013/024779 and International Publication No. WO2015/137486. The documents describe a method of reacting a naphthol, a biphenol or the like with an aldehyde, a ketone or the like in the presence of an acid catalyst.

### [Resin Having Structure Represented by Formula (2A)]

Examples of the resin used in the present embodiment include a resin having a structure represented by the following formula (2A).

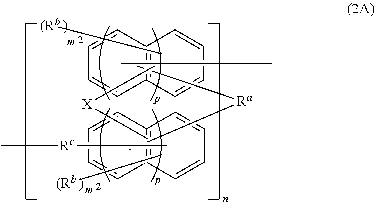

In the formula (2A), X, R^{a}, R^{b}, n and p are the same as defined in the formula (1A). Herein, at least one R^{b} represents a group comprising one or more selected from a hydroxyl group and a thiol group.

R^{c} represents a single bond or an alkylene group having 1 to 40 carbon atoms. The alkylene group may be either linear or branched.

Each m² is independently an integer of 0 to 8; and all m² cannot be 0 at the same time.

The resin having a structure represented by the formula (2A) is preferably a resin having a structure represented by the following formula (2) from the viewpoint of ease of production.

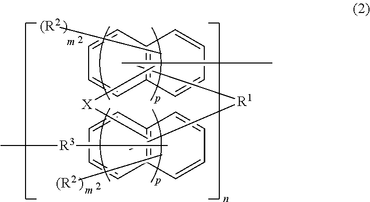

In the formula (2), X, R¹, R², n and p are the same as defined in the formula (1). Herein, at least one R² represents one selected from a hydroxyl group and a thiol group.

R³ is the same as R^{c} defined in the formula (2A)

m² is the same as defined in the formula (2A). Herein, all m² cannot be 0 at the same time.

The resin having a structure represented by the formula (2) is preferably a resin having a structure represented by the following formula (2-1) from the viewpoint of improvement in heat resistance.

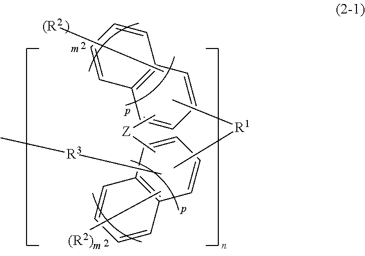

In the formula (2-1), Z is the same as defined in the formula (1-1) and represents an oxygen atom or a sulfur atom.

R¹, R², R³, m², p and n are the same as defined in the formula (2). Herein, at least one R² represents one selected from a hydroxyl group and a thiol group, and all m² cannot be 0 at the same time.

In addition, the resin having a structure represented by the formula (2) preferably has a structure represented by the following formula (4).

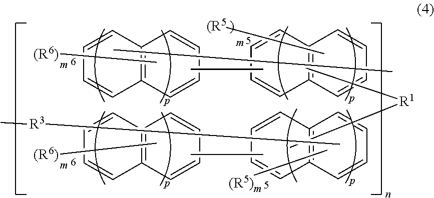

In the formula (4), R¹, R⁵, R⁶, m⁵, m⁶, p and n are the same as defined in the formula (3).

R³ is the same as defined in the formula (2).

Herein, at least one selected from R⁵ and R⁶ represents one selected from a hydroxyl group and a thiol group, and all of m⁵ and m⁶ cannot be 0 at the same time.

### [Resin Having Structure Represented by Formula (2A′)]

In the present embodiment, the resin having a structure represented by the formula (2A) is preferably a resin having a structure represented by the following formula (2A′) from the viewpoint of solubility in organic solvents.

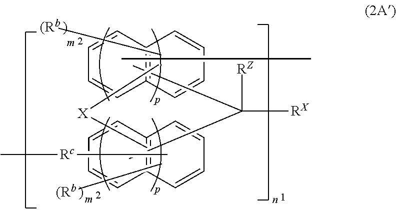

In the formula (2A′), R^{b}, X, m² and p are the same as defined in the formula (2A); and R^{x}, R^{z} and n¹ are the same as defined in the formula (1A′).

The resin having a structure represented by the formula (2A′) is preferably a resin having a structure represented by the following formula (2′) from the viewpoint of ease of production.

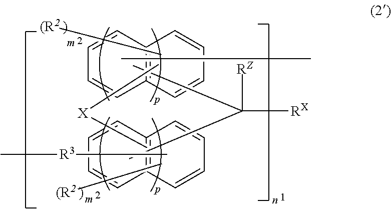

(wherein, R^{x}, R^{z}, X, m², n¹ and p are the same as defined in the formula (2A); and R² and R³ are the same as defined in the formula (2).)

The resin having a structure represented by the formula (2′) is preferably a resin having a structure represented by the following formula (2-1′) from the viewpoint of improvement in heat resistance.

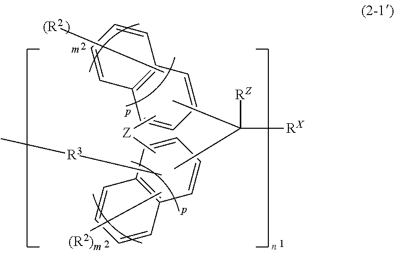

(wherein, R^{x}, R^{z}, R², R³, m², n¹ and p are the same as defined in the formula (2′); and Z is the same as defined in the formula (2-1).)

In addition, the resin having a structure represented by the formula (2′) preferably has a structure represented by the following formula (4′).

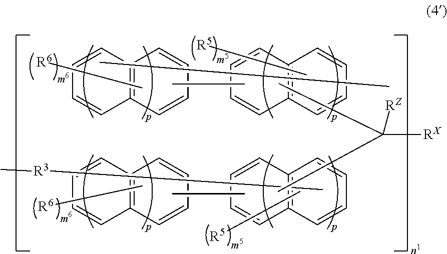

(wherein, R^{x}, R^{z}, n¹ and p are the same as defined in the formula (2′); and R⁵, R⁶, m⁵ and m⁶ are the same as defined in the formula (4).)

### [Method for Preparing Resin Having Structure Represented by Formula (2A) and Resin Having Structure Represented by Following Formula (2A′)]

The resin having a structure represented by the formula (2A) and the resin having a structure represented by following formula (2A′), which are used in the present embodiment, can be appropriately synthesized by applying a known method without particular limitation. These resins can be produced, for example, by the methods described in International Publication No. WO2013/024779 and International Publication No. WO2015/137486. The documents describe a method of reacting a compound, which has been obtained by reacting a naphthol, a biphenol or the like with an aldehyde, a ketone or the like in the presence of an acid catalyst, with a compound having crosslinking reactivity and then oligomerizing or polymerizing it.

### [Preparation Step of Solution]

The solution to be purified for use in the present embodiment comprises one or more materials selected from a compound represented by the formula (1A) and a resin having a structure represented by the formula (2A), and a solvent described below. The solution may also contain various surfactants, various crosslinking agents, various acid generators, various stabilizers and the like.

Examples of the solvent to be used in the present embodiment include, but not particularly limited to, an organic solvent that can be safely applied to a semiconductor manufacturing process. The amount of the solvent to be used is preferably usually 1 to 100 times by mass based on the amount of the material to be purified from the viewpoint of improvement in solubility and ease of collection of solids after purification. It is more preferably 5 to 50 times by mass, and further preferably 10 to 50 times by mass.

Specific examples of the solvent to be used include, but not limited to the following: ethers such as ethyl ether, isopropyl ether, n-butyl ether, hexyl ether, 2-ethylhexyl ether, ethylene oxide, 1,2-propylene oxide, dioxolane, 4-methyldioxolane, dioxane, dimethyl dioxane, ethylene glycol monomethyl ether, ethylene glycol monoethyl ether, ethylene glycol diethyl ether, ethylene glycol monobutyl ether, ethylene glycol mono-n-hexyl ether, ethylene glycol monophenyl ether, ethylene glycol mono-2-ethyl butyl ether, ethylene glycol dibutyl ether, diethylene glycol monomethyl ether, diethylene glycol monoethyl ether, diethylene glycol diethyl ether, diethylene glycol monobutyl ether, diethylene glycol dibutyl ether, diethylene glycol mono-n-hexyl ether, ethoxytriglycol, tetraethylene glycol dibutyl ether, propylene glycol monomethyl ether (PGME), dipropylene glycol methyl ether, tripropylene glycol methyl ether, propylene glycol monopropyl ether, tetrahydrofuran and 2-methyltetrahydrofuran; monoalcohols such as methanol, ethanol, n-propanol, i-propanol, n-butanol, i-butanol, sec-butanol, t-butanol, n-pentanol, i-pentanol, 2-methylbutanol, sec-pentanol, t-pentanol, 3-methoxybutanol, n-hexanol, 2-methylpentanol, sec-hexanol, 2-ethylbutanol, sec-heptanol, 3-heptanol, n-octanol, 2-ethylhexanol, sec-octanol, nonyl alcohol, 2,6-dimethyl-4-heptanol, n-decanol, sec-undecyl alcohol, trimethyl nonyl alcohol, sec-tetradecyl alcohol, sec-heptadecyl alcohol, phenol, cyclohexanol, methylcyclohexanol, 3,3,5-trimethylcyclohexanol, benzyl alcohol, phenyl methyl carbinol, diacetone alcohol and cresol; esters such as diethyl carbonate, methyl acetate, ethyl acetate, γ-butyrolactone, γ-valerolactone, n-propyl acetate, i-propyl acetate, n-butyl acetate, i-butyl acetate, sec-butyl acetate, n-pentyl acetate, sec-pentyl acetate, 3-methoxybutyl acetate, methylpentyl acetate, 2-ethylbutyl acetate, 2-ethylhexyl acetate, benzyl acetate, cyclohexyl acetate, methyl cyclohexyl acetate, nonyl acetate, methyl acetoacetate, ethyl acetoacetate, ethylene glycol monomethyl ether acetate, ethylene glycol monoethyl ether acetate, propylene glycol monomethyl ether acetate, diethylene glycol monomethyl ether acetate, diethylene glycol monoethyl ether acetate, diethylene glycol monobutyl ether acetate, glycol diacetate, methoxytriglycol acetate, ethyl propionate, n-butyl propionate, i-pentyl propionate, methyl methoxypropionate, ethyl ethoxypropionate, diethyl oxalate, di-n-butyl oxalate, methyl lactate, ethyl lactate, n-butyl lactate, n-pentyl lactate, diethyl malonate, dimethyl phthalate and diethyl phthalate; ketones such as acetone, methyl ethyl ketone, methyl n-propyl ketone, methyl n-butyl ketone, diethyl ketone, methyl isobutyl ketone, methyl n-pentyl ketone, ethyl butyl ketone, methyl hexyl ketone, diisobutyl ketone, trimethylnonanone, cyclohexanone, methyl cyclohexanone, 2,4-pentanedione, acetonylacetone, acetophenone and N-methyl pyrrolidone; glycol ether acetates such as ethylene glycol monoethyl ether acetate, ethylene glycol monobutyl ether acetate, propylene glycol monomethyl ether acetate (PGMEA) and propylene glycol monoethyl ether acetate; nitrogen compound-based solvents such as N-methylformamide, N, N-dimethylformamide, N, N-diethylformamide, acetamide, N-methylacetamide, N, N-dimethylacetamide, N-methylpropionamide and N-methylpyrrolidone; aliphatic hydrocarbons such as n-hexane and n-heptane; aromatic hydrocarbons such as toluene and xylene; and halogenated hydrocarbons such as methylene chloride and chloroform.

Among them, ethyl acetate, butyl acetate, methyl isobutyl ketone, propylene glycol monomethyl ether (PGME), propylene glycol monomethyl ether acetate (PGMEA), cyclopentanone and cyclohexanone are preferable. These solvents can be used alone or can be used as a mixture of two or more. They are preferable in terms of processability and ease of management of the amount to be charged.

### [Step of Purification of Solution (Liquid Passing Step]

In the step of passing a liquid through a filter in the present embodiment, a filter used for removing metal components in a solution containing the material and solvent can be generally one commercially available for liquid filtration. The filtration accuracy of the filter is not particularly limited, but the nominal pore size of the filter is preferably 0.2 μm or less, more preferably less than 0.2 μm, further preferably 0.1 μm or less, still further preferably less than 0.1 μm, and furthermore preferably 0.05 μm or less. The lower limit of the nominal pore size of the filter is not particularly limited, but is usually 0.005 μm. As used herein, the term “nominal pore size” refers to the pore size nominally used to indicate the separation performance of the filter, which is determined, for example, by any method specified by the filter manufacturer, such as a bubble point test, a mercury intrusion test or a standard particle trapping test. When using a commercially available product, the nominal pore size is a value described in the manufacturer's catalog data. The nominal pore size of 0.2 μm or less makes it possible to effectively reduce the contents of the metal components after passing the solution through the filter once. Particularly, the content of chromium (Cr) can be reduced to preferably 50 ppb or less, more preferably 20 ppb or less and further more preferably 5 ppb or less, based on the mass of the used material to be purified. In the present embodiment, the step of passing a liquid through a filter may be performed twice or more to reduce the content of each metal component in the solution.

Examples of the filter to be used include a hollow fiber membrane filter, a membrane filter, a pleated membrane filter, and a filter filled with a filter medium such as a non-woven fabric, cellulose or diatomaceous earth. Among the above, the filter is preferably one or more selected from the group consisting of a hollow fiber membrane filter, a membrane filter and a pleated membrane filter. In addition, it is particularly preferable to use a hollow fiber membrane filter, in particular due to its high precision filtration accuracy and its higher filtration area than other forms.

Examples of a material for the filter include a polyolefin such as polyethylene or polypropylene; a polyethylene-based resin having a functional group having an ion exchange capacity provided by graft polymerization; a polar group-containing resin such as polyamide, polyester or polyacrylonitrile; and a fluorine-containing resin such as fluorinated polyethylene such as (PTFE). Among the above, the filter is preferably made of one or more filter media selected from the group consisting of a polyamide, a polyolefin resin and a fluorocarbon resin. These filters are preferably employed, because the concern of metal elution from the filter medium tends to be reduced as compared to, for example, filters of sintered metal materials. Furthermore, a polyamide medium is particularly preferable from the viewpoint of the reduction effect of heavy metals such as chromium.

Examples of the polyamide filter include (hereinafter described under the trade name), but not limited to: Polyfix nylon series available from KITZ MICROFILTER CORPORATION; Ultipleat P-Nylon 66 and Ultipor N66 available from Nihon Pall Ltd.; and LifeASSURE PSN series and LifeASSURE EF series available from 3M Company.

Examples of polyolefin-based filter include, but are not limited to: Ultipleat PE Clean and Ion Clean available from Nihon Pall Ltd.; Protego series, Microgard Plus HC10 and Optimizer D available from Entegris Japan Co., Ltd.

Examples of the polyester-based filter include, but are not limited to: Geraflow DFE available from Central Filter Mfg. Co., Ltd.; and a pleated type PMC available from Nihon Filter Co., Ltd.

Examples of the polyacrylonitrile-based filter include, but are not limited to: Ultrafilters AIP-0013D, ACP-0013D and ACP-0053D available from Advantec Toyo Kaisha, Ltd.

Examples of the fluororesin-based filter include, but are not limited to: Emflon HTPFR available from Nihon Pall Ltd.; and LifeASSURE FA series available from 3M Company.

These filters may be used alone or in combination of two or more thereof.

The filter may also contain an ion exchanger such as a cation-exchange resin, or a cation charge controlling agent that causes a zeta potential in an organic solvent solution to be filtered.

Examples of the filter containing an ion exchanger, but are limited to: Protego series available from Entegris Japan Co., Ltd.; and KURANGRAFT available from Kurashiki Textile Manufacturing Co., Ltd.

Examples of the filter containing a material having a positive zeta potential such as a cationic polyamidepolyamine-epichlorohydrin resin include (hereinafter described under the trade name), but not limited to: Zeta Plus 40QSH and Zeta Plus 020GN and LifeASSURE EF series available from 3M company.

In addition, at least one packing member such as an O-ring included in a connection joint and a housing of the filter is made of a perfluoro rubber or a perfluoro elastomer, and all these members are preferably composed of a material selected from a fluorine-containing resin, a perfluoro rubber or a perfluoro elastomer. In addition, the packing member is particularly preferably composed of a material selected from a perfluoro rubber and a perfluoro elastomer. Use of these members tends to sufficiently reduce the contents of metal components.

Too high temperature during the purification of a solution comprising the above-described material is not preferable because it may lead to liberation of a volatile acid due to the hydrolysis of the material, depending on the type of the solvent. In contrast, too low temperature is not efficient due to the low solubility of the material to be purified. The temperature to be selected may be usually 0 to 40° C., preferably 5 to 30° C. and particularly preferably 10 to 25° C.

The purification method of the present embodiment may further comprise a purification step other than the step of passing a liquid through a filter.

Water included in the thus-obtained solution can be easily removed by an operation such as evaporation under reduced pressure. A solvent may also be added if necessary to adjust the solution to any concentration.

Only the material to be purified can be obtained from a solution comprising the material to be purified and a solvent by a known method such as removal under reduced pressure or separation by reprecipitation or a combination thereof. If necessary, a known process such as a concentration operation, a filtration operation, a centrifugation operation or a drying operation can be performed.

The purification method of the present embodiment is preferably performed in an atmosphere with an oxygen concentration of less than 20%. That is, the oxygen concentration in the atmosphere in contact with a solution comprising the material to be purified and a solvent is preferably adjusted to less than 20%, and maintained less than 20% in a series of operations before the step of passing the solution through a filter. The oxygen concentration in the atmosphere is more preferably less than 20% from the stage after preparation of the solution comprising the material to be purified and the solvent to the step of passing the solution through the filter.

The oxygen concentration is more preferably less than 10%, further preferably less than 5% and particularly preferably less than 1%. The oxygen concentration of less than 20% can inhibit the material to be purified from be altered, and tends to provide a more highly pure material.

The oxygen concentration can be reduced by a known method including, but not particularly limited to, a method of flowing a nitrogen gas through a column or tank to be used for purification or depressurizing the column or tank followed by introduction of a nitrogen gas so as to perform gas replacement. It is preferably performed by depressurizing the column or tank followed by introduction of a nitrogen gas because of simplicity and reliability.

The oxygen concentration can be confirmed by a known method including, but not particularly limited to, a method of flowing a nitrogen gas through the tank to be used for purification and then measuring the oxygen concentration in the gas discharged from the vent with an oximeter. The oximeter may be also provided into the tank to be used for purification.

### (Method for Producing Composition)

The method for producing a composition according to the present embodiment is a method for producing a composition comprising one or more materials selected from a compound represented by the following formula (1A) and a resin having a structure represented by the following formula (2A), 99 ppb or less of Na, less than 60 ppb of Fe, less than 80 ppb of Cr and less than 70 ppb of Sn, comprising: a step of preparing a solution comprising a solvent and a precursor composition comprising the material and more than 99 ppb of Na, 60 ppb or more of Fe, 80 ppb or more of Cr and 70 ppb or more of Sn; and a step of passing the solution through a filter to reduce the contents of Na, Fe, Cr and Sn in the solution to 99 ppb or less, less than 60 ppb, less than 80 ppb and less than 70 ppb, respectively. That is, the precursor composition in the present embodiment can be also described to be a mixture of the material in the present embodiment and impurities (all components other than the material of interest), and can be subjected to the purification in the present embodiment to provide the composition of the present embodiment.

As described above, the compound represented by the formula (1A) and the resin having a structure represented by the formula (2A) to be used in the present embodiment and the solvent are the same as the compound, the resin and the solvent in the purification method of the present embodiment. The step of passing the solution through a filter can be performed as the liquid passing step in the purification method of the present embodiment.

### EXAMPLES

Hereinafter, the present embodiment will be more specifically described with reference to Examples. The present embodiment, however, is not limited to these Examples.

The ¹H-NMR was measured under the following conditions with an “Advance 600 II spectrometer” available from Bruker Corporation.

Frequency: 400 MHz

Solvent: d6-DMSO

Internal standard: TMS

Measurement temperature: 23° C.

### (Synthesis Example 1) Synthesis of BisN-1

Into a vessel having an inner volume of 500 mL, equipped with a stirrer, a condenser and a burette, were charged 20.0 g (200 mmol) of 1,4-dihydroxybenzene (a reagent available from KANTO CHEMICAL CO., INC.), 18.2 g (100 mmol) of 4-biphenyl aldehyde (available from MITSUBISHI GAS CHEMICAL COMPANY, INC.) and 100 mL of 1,4-dioxane, and 5 mL of 95% sulfuric acid was added thereto and the mixture was stirred at 100° C. for 6 hours to perform a reaction. Next, the reaction liquid was neutralized with a 24% aqueous sodium hydroxide solution, and 50 g of pure water was then added thereto to precipitate a reaction product, which was cooled to room temperature followed by filtration for separation. The resulting solid was dried and then subjected to separation and purification by column chromatography to provide 20.6 g of a target compound (BisN-1) represented by the following formula.

Herein, the following peaks were observed by 400 MHz-¹H-NMR, and it was confirmed that the compound had a chemical structure of the following formula.

¹H-NMR: (d-DMSO, Internal reference TMS)

δ (ppm) 9.4 (2H, O—H), 7.2-8.1 (13H, Ph-H), 6.5 (1H, C—H)

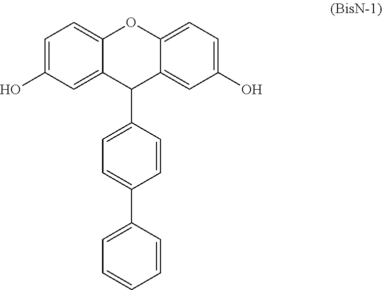 [SMILES: Oc1ccc2c(c1)C(c1ccc(-c3ccccc3)cc1)c1cc(O)ccc1O2]

### (Synthesis Example 2) Synthesis of BisN-2

Into a vessel having an inner volume of 500 mL, equipped with a stirrer, a condenser and a burette, were charged 32.0 g (20 mmol) of 2,6-naphthalenediol (a reagent available from Sigma-Aldrich), 18.2 g (100 mmol) of 4-biphenyl aldehyde (available from MITSUBISHI GAS CHEMICAL COMPANY, INC.), and 200 mL of 1,4-dioxane, 10 mL of 95% sulfuric acid was added thereto and the mixture was stirred at 100° C. for 6 hours to perform a reaction. Next, the reaction liquid was neutralized with a 24% aqueous sodium hydroxide solution, and 100 g of pure water was added thereto to precipitate a reaction product, which was cooled to room temperature followed by filtration for separation. The resulting solid was filtered, dried and then subjected to separation and purification by column chromatography to provide 25.5 g of a target compound (BisN-2) represented by the following formula.

Herein, the following peaks were observed by 400 MHz-¹H-NMR, and it was confirmed that the compound had a chemical structure of the following formula. In addition, it was confirmed from a doublet signal of protons at 3- and 4-positions that 2,6-dihydroxynaphthol was substituted at 1-position.

¹H-NMR: (d-DMSO, Internal reference TMS)

δ (ppm) 9.7 (2H, O—H), 7.2-8.5 (19H, Ph-H), 6.6 (1H, C—H)

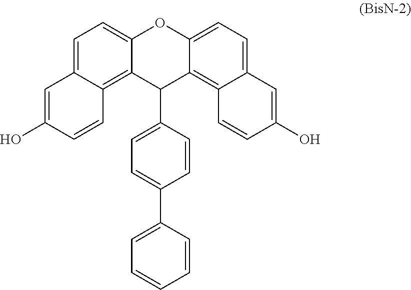 [SMILES: Oc1ccc2c3c(ccc2c1)Oc1ccc2cc(O)ccc2c1C3c1ccc(-c2ccccc2)cc1]

### (Synthesis Example 3) Synthesis of RBisN-2

Into a vessel having an inner volume of 500 mL, equipped with a stirrer, a condenser and a burette, were charged 50 g (105 mmol) of BisN-2, 3.5 g (210 mmol) of paraformaldehyde, 50 mL of glacial acetic acid and 200 mL of PGME, 30 mL of 95% sulfuric acid was added thereto and the mixture was stirred at 100° C. for 6 hours to perform a reaction. Next, the reaction liquid was concentrated, and 1000 mL of methanol was added thereto to precipitate a reaction product, which was cooled to room temperature followed by filtration for separation. The resulting solid was filtered, dried and then subjected to separation and purification by column chromatography to provide 35.0 g of a target resin (RBisN-2) having a structure represented by the following formula.

The molecular weight in terms of polystyrene with respect to the resulting resin was measured by the above method, and as a result, Mn was 778, Mw was 1793 and Mw/Mn was 2.30.

NMR measurement of the resulting resin was performed under the above measurement conditions, and the following peaks were observed. It was confirmed that the resin had a chemical structure of the following formula.

δ (ppm) 9.7 (2H, O—H), 7.2-8.5 (17H, Ph-H), 6.6 (1H, C—H), 4.1 (2H, —CH₂)

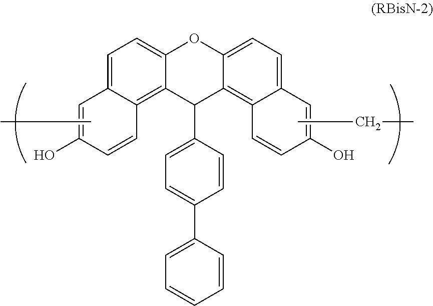

### (Synthesis Example 4) Synthesis of CH—BisN

Into a vessel having an inner volume of 500 mL, equipped with a stirrer, a condenser and a burette, were charged 32.0 g (20 mmol) of 2,7-naphthalenediol (a reagent available from Sigma-Aldrich), 18.8 g (100 mmol) of cyclohexyl benzaldehyde (available from MITSUBISHI GAS CHEMICAL COMPANY, INC.), and 200 mL of 1,4-dioxane, 10 mL of 95% sulfuric acid was added thereto and the mixture was stirred at 100° C. for 6 hours to perform a reaction. Next, the reaction liquid was neutralized with a 24% aqueous sodium hydroxide solution, and 100 g of pure water was added thereto to precipitate a reaction product, which was cooled to room temperature followed by filtration for separation. The resulting solid was dried and then subjected to separation and purification by column chromatography to provide 30.5 g of a target compound (CH—BisN) represented by the following formula.

Herein, the following peaks were observed by 400 MHz-¹H-NMR, and it was confirmed that the compound had a chemical structure of the following formula.

¹H-NMR: (d-DMSO, Internal reference TMS)

δ (ppm) 9.7 (2H, O—H), 7.2-8.0 (14H, Ph-H), 6.2 (1H, C—H), 3.4-3.6 (11H, C—H)

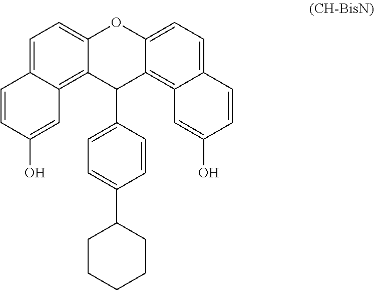 [SMILES: Oc1ccc2ccc3c(c2c1)C(c1ccc(C2CCCCC2)cc1)c1c(ccc2ccc(O)cc12)O3]

### (Synthesis Example 5) Synthesis of CAX-1

A glass vessel having an inner volume of 1 L equipped with a stirrer, a condenser and a burette was prepared. Into this vessel were charged 89.0 g (400 mmol) of N-ethylcarbazole-3-carbaldehyde (available from NISSHOKU TECHNO FINE CHEMICAL CO., LTD), 128.0 g (800 mmol) of 2,6-dihydroxynaphthalene (a reagent available from Tokyo Chemical Industry Co., Ltd.) and 300 mL of 1,4-dioxane (a reagent available from KANTO CHEMICAL CO., INC.), and 19.5 g (105 mmol) of p-toluenesulfonic acid (a reagent available from KANTO CHEMICAL CO., INC.) was added thereto to prepare a reaction liquid. The reaction liquid was stirred at 90° C. for 6 hours to perform a reaction. Next, the reaction liquid was neutralized with a 24% aqueous sodium hydroxide solution (a reagent available from KANTO CHEMICAL CO., INC.) and concentrated, and 100 mL of n-heptane (a reagent available from KANTO CHEMICAL CO., INC.) was added thereto to precipitate a reaction product, which was cooled to room temperature followed by filtration for separation. The solid obtained by filtration was dried and then subjected to separation and purification by column chromatography to provide 20.2 g of a target compound (CAX-1) represented by the following formula.

Herein, the following peaks were observed by 400 MHz-¹H-NMR, and it was confirmed that the compound had a chemical structure of the following formula.

¹H-NMR: (d-DMSO, Internal reference TMS)

δ (ppm) 9.9 (2H, O—H), 7.0-8.3 (17H, Ph-H), 6.2 (1H, C—H), 4.2 (2H, CH₂), 1.2 (3H, CH₃)

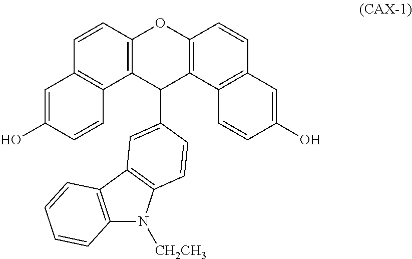 [SMILES: CCn1c2ccccc2c2cc(C3c4c(ccc5cc(O)ccc45)Oc4ccc5cc(O)ccc5c43)ccc21]

### (Synthesis Example 6) Synthesis of BiF-1

A vessel having an inner volume of 1 L equipped with a stirrer, a condenser and a burette was prepared. Into this vessel were charged 150 g (800 mmol) of 4,4-biphenol (a reagent available from Tokyo Chemical Industry Co., Ltd.), 75 g (410 mmol) of 4-biphenyl aldehyde (available from MITSUBISHI GAS CHEMICAL COMPANY, INC.) and 300 mL of propylene glycol monomethyl ether, and 19.5 g (105 mmol) of p-toluenesulfonic acid (a reagent available from KANTO CHEMICAL CO., INC.) was added thereto to prepare a reaction liquid. The reaction liquid was stirred at 90° C. for 3 hours to perform a reaction. Next, the reaction liquid was neutralized with a 24% aqueous sodium hydroxide solution, and 100 g of distilled water was added thereto to precipitate a reaction product, which was cooled to 5° C. followed by filtration for separation. The solid obtained by filtration was dried and then subjected to separation and purification by column chromatography to provide 25.8 g of a target compound (BiF-1) represented by the following formula.

Herein, the following peaks were observed by 400 MHz-¹H-NMR, and it was confirmed that the compound had a chemical structure of the following formula.

¹H-NMR: (d-DMSO, Internal reference TMS)

δ (ppm) 9.4 (4H, O—H), 6.8-7.8 (22H, Ph-H), 6.2 (1H, C—H)

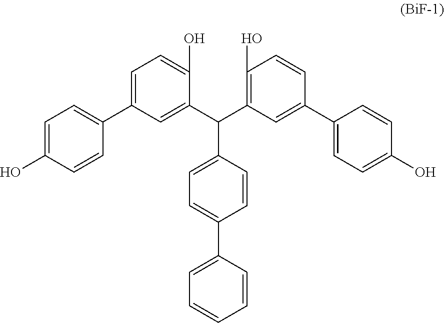 [SMILES: Oc1ccc(-c2ccc(O)c(C(c3ccc(-c4ccccc4)cc3)c3cc(-c4ccc(O)cc4)ccc3O)c2)cc1]

### (Synthesis Example 7) Synthesis of BiF—I-1

A vessel having an inner volume of 1 L equipped with a stirrer, a condenser and a burette was prepared. Into this vessel were charged 150 g (800 mmol) of 4,4-biphenol (a reagent available from Tokyo Chemical Industry Co., Ltd.), 75 g (325 mmol) of 4-iodobenzaldehyde (available from Tokyo Chemical Industry Co., Ltd.) and 300 mL of propylene glycol monomethyl ether, and 19.5 g (105 mmol) of p-toluenesulfonic acid (a reagent available from KANTO CHEMICAL CO., INC.) was added thereto to prepare a reaction liquid. The reaction liquid was stirred at 90° C. for 6 hours to perform a reaction. Next, the reaction liquid was neutralized with a 24% aqueous sodium hydroxide solution, and 100 g of distilled water was added thereto to precipitate a reaction product, which was cooled to room temperature followed by filtration for separation. The solid obtained by filtration was dried and then subjected to separation and purification by column chromatography to provide 24.3 g of a target compound (BiF—I-1) represented by the following formula.

Herein, the following peaks were observed by 400 MHz-¹H-NMR, and it was confirmed that the compound had a chemical structure of the following formula.

¹H-NMR: (d-DMSO, Internal reference TMS)

δ (ppm) 9.4 (4H, O—H), 6.8-7.8 (18H, Ph-H), 6.2 (1H, C—H)

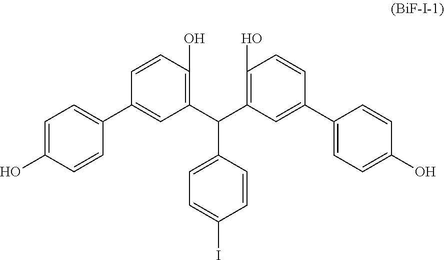 [SMILES: Oc1ccc(-c2ccc(O)c(C(c3ccc(I)cc3)c3cc(-c4ccc(O)cc4)ccc3O)c2)cc1]

### Synthesis Examples 8 and 9

Each target product was obtained as in Synthesis Example 2 except that 2,6-naphthalenediol and 4-biphenylcarboxaldehyde, which were the raw materials in Synthesis Example 2, were changed to the raw material 1 and the raw material 2 in Table 1; 1.5 mL of water, 73 mg (0.35 mmol) of dodecyl mercaptan and 2.3 g (22 mmol) of 37% hydrochloric acid were added; and the reaction temperature was changed to 55° C. Each product was identified by ¹H-NMR. The results are shown in Table 2.

TABLE 1

| Synthesis | | | |
|---|---|---|---|
| Example | Raw material 1 | Raw material 2 | Product |
| 8 | Resorcinol | Benzaldehyde | P-6 |
| 9 | Resorcinol | 4-Cyclohexylbenzaldehyde | P-7 |

TABLE 2

| Synthesis | Compound | |
|---|---|---|
| Example | name | 1H-NMR |
| 8 | P-6 | δ (ppm) 9.3-9.4 (4H, O—H), |
| | | 6.6-7.2 (11H, Ph—H), 6.2 (1H, C—H) |
| 9 | P-7 | δ (ppm) 9.2-9.4 (4H, O—H), |
| | | 6.4-7.2 (10H, Ph—H), 1.4-1.9 (10H, C—H2), |
| | | 2.7 (1H, C—H), 2.5 (1H, C—H) |

From the above results, it was confirmed that the products had chemical structures of the following formulae (P-6) and (P-7), respectively.

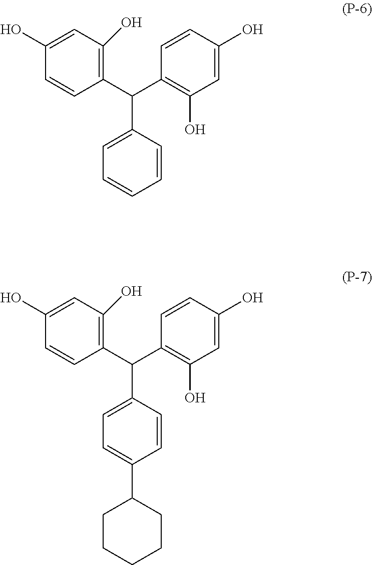 [SMILES: Oc1ccc(C(c2ccccc2)c2ccc(O)cc2O)c(O)c1 ; Oc1ccc(C(c2ccc(C3CCCCC3)cc2)c2ccc(O)cc2O)c(O)c1]

### (Synthesis Example 10) Synthesis of BiN-1

Ten gram (69.0 mmol) of 2-naphthol (a reagent available from Sigma-Aldrich) was molten at 120° C. in a vessel having an inner volume of 300 mL equipped with a stirrer, a condenser and a burette, 0.27 g of sulfuric acid was then charged thereinto, and 2.7 g (13.8 mmol) of 4-acetyl biphenyl (a reagent available from Sigma-Aldrich) was added thereto. The contents were stirred at 120° C. for 6 hours to perform a reaction so as to provide a reaction liquid. Next, 100 mL of N-methyl-2-pyrrolidone (available from KANTO CHEMICAL CO., INC.) and 50 mL of pure water were added to the reaction liquid, and the mixture was then extracted with ethyl acetate. Thereafter, pure water was added thereto, and the mixture was subjected to liquid separation until it became neutral and subjected to concentration to provide a solution.

The resulting solution was subjected to separation by column chromatography to provide 1.0 g of a target compound (BiN-1) represented by the following formula (BiN-1).

The molecular weight of the resulting compound (BiN-1) was measured by the above-described method, and as a result, it was 446.

NMR measurement of the resulting compound (BiN-1) was performed under the above measurement conditions, and the following peaks were observed. It was confirmed that the compound had a chemical structure of the following formula (BiN-1).

δ (ppm) 9.69 (2H, O—H), 7.01-7.67 (21H, Ph-H), 2.28 (3H, C—H)

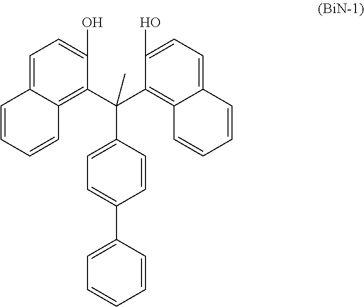 [SMILES: CC(c1ccc(-c2ccccc2)cc1)(c1c(O)ccc2ccccc12)c1c(O)ccc2ccccc12]

### (Synthesis Example 11) Synthesis of BiP-1

The reaction was performed as in Synthesis Example 10 except that 2,2′-biphenol was used instead of 2-naphthol, to provide 0.1 g of a target compound represented by the following formula (BiP-1).

The molecular weight of the resulting compound (BiP-1) was measured by the above-described method, and as a result, it was 466.

NMR measurement of the resulting compound (BiP-1) was performed under the above measurement conditions, and the following peaks were observed. It was confirmed that the compound had a chemical structure of the following formula (BiP-1).

δ (ppm) 9.40 (4H, O—H), 6.80-7.80 (23H, Ph-H), 2.25 (3H, C—H)

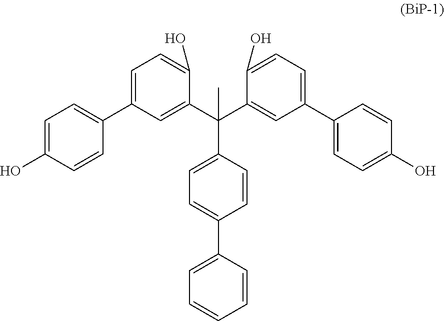 [SMILES: CC(c1ccc(-c2ccccc2)cc1)(c1cc(-c2ccc(O)cc2)ccc1O)c1cc(-c2ccc(O)cc2)ccc1O]

### Synthesis Examples 12 to 19

Each target product was obtained as in Synthesis Example 11 except that 2-naphthol and 4-acetylbiphenyl, which were the raw materials in Synthesis Example 10, were changed as shown in Table 3. Each product was identified by 1H-NMR. The results are shown in Table 4.

TABLE 3

| Synthesis | | | |
|---|---|---|---|
| Example | Raw material 1 | Raw material 2 | Product |
| 12 | 2,6-Dihydroxynaphthalene | 4-Acetyl biphenyl | BiN-2 |
| 13 | 2,7-Dihydroxynaphthalene | 4-Acetyl biphenyl | BiN-3 |
| 14 | 2,6-Dihydroxynaphthalene | 4′-Cyclohexyl | BiN-4 |
| | | acetophenone | |
| 15 | p-Phenylphenol | 4-Acetyl biphenyl | BiP-2 |
| 16 | 2,2′-Dihydroxybiphenyl | 4-Acetyl biphenyl | BiP-3 |
| 17 | 2,2′-Dihydroxybiphenyl | 4′-Cyclohexyl | BiP-4 |
| | | acetophenone | |
| 18 | Phenol | 4-Acetyl biphenyl | P-1 |
| 19 | Phenol | 4′-Cyclohexyl | P-2 |
| | | acetophenone | |
| 20 | Resorcinol | 4-Acetyl biphenyl | P-3 |
| 21 | Resorcinol | 4′-Cyclohexyl | P-4 |
| | | acetophenone | |

TABLE 4

| Synthesis | Compound | |
|---|---|---|
| Example | name | 1H-NMR |
| 12 | BiN-2 | δ (ppm) 9.2-9.7 (4H, O—H), 6.8-7.9 (19H, Ph—H), 2.5 (3H, C—H₃) |
| 13 | BiN-3 | δ (ppm) 9.2-9.7 (4H, O—H), 6.9-7.8 (19H, Ph—H), 2.5 (3H, C—H₃) |
| 14 | BiN-4 | δ (ppm) 9.2-9.7 (4H, O—H), 6.8-7.8 (14H, Ph—H), 2.5 (3H, C—H₃), |
| | | 1.4-1.9 (10H, C—H₂), 2.7 (1H, C—H) |
| 15 | BiP-2 | δ (ppm) 9.7 (4H, O—H), 6.8-7.8 (23H, Ph—H), 2.3 (3H, C—H₃) |
| 16 | BiP-3 | δ (ppm) 9.0 (4H, O—H), 7.0-7.8 (23H, Ph—H), 2.3 (3H, C—H₃) |
| 17 | BiP-4 | δ (ppm) 9.0 (4H, O—H), 7.0-7.8 (18H, Ph—H), 2.3 (3H, C—H₃), |
| | | 1.4-1.9 (10H, C—H₂), 2.7 (1H, C—H) |
| 18 | P-1 | δ (ppm) 9.1 (2H, O—H), 6.6-7.8 (17H, Ph—H), 2.3 (3H, C—H₃) |
| 19 | P-2 | δ (ppm) 9.1 (2H, O—H), 6.6-7.2 (12H, Ph—H), 2.3 (3H, C—H₃), |
| | | 1.4-1.9 (10H, C—H2), 2.7 (1H, C—H) |

From the above results, it was confirmed that the products had chemical structures of the following formulae (BiN-2) to (P-2), respectively.

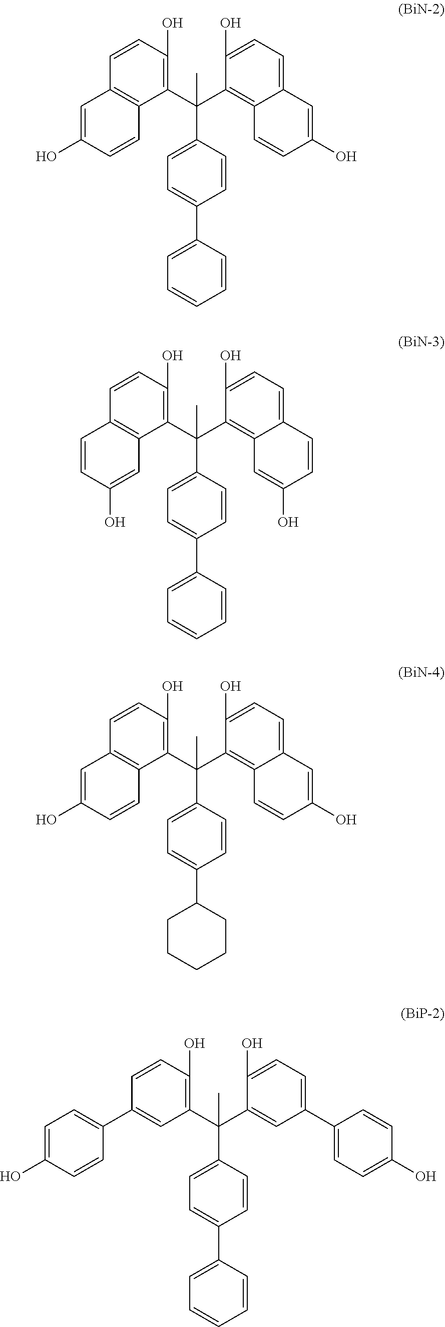 [SMILES: CC(c1ccc(-c2ccccc2)cc1)(c1c(O)ccc2cc(O)ccc12)c1c(O)ccc2cc(O)ccc12 ; CC(c1ccc(-c2ccccc2)cc1)(c1c(O)ccc2ccc(O)cc12)c1c(O)ccc2ccc(O)cc12 ; CC(c1ccc(C2CCCCC2)cc1)(c1c(O)ccc2cc(O)ccc12)c1c(O)ccc2cc(O)ccc12 ; CC(c1ccc(-c2ccccc2)cc1)(c1cc(-c2ccc(O)cc2)ccc1O)c1cc(-c2ccc(O)cc2)ccc1O]

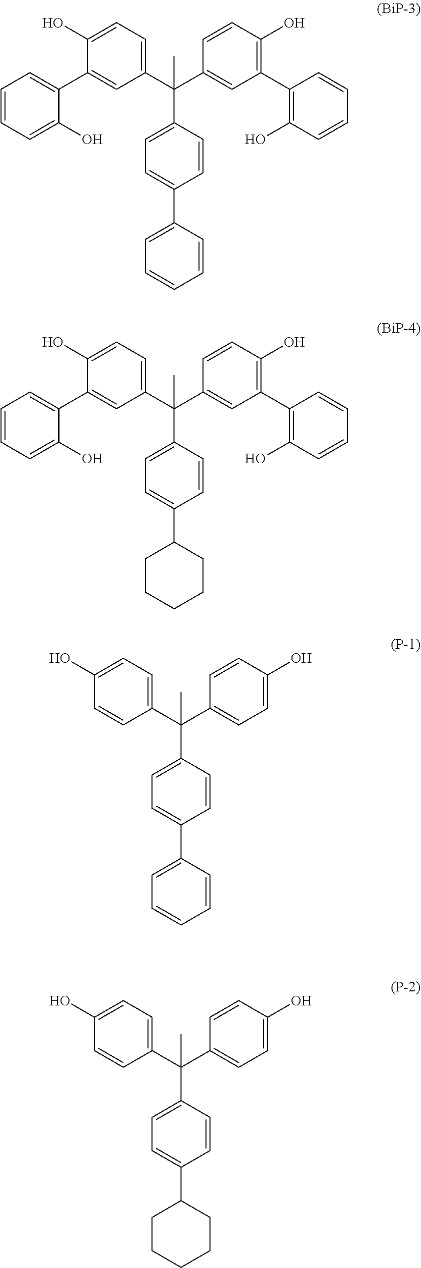 [SMILES: CC(c1ccc(-c2ccccc2)cc1)(c1ccc(O)c(-c2ccccc2O)c1)c1ccc(O)c(-c2ccccc2O)c1 ; CC(c1ccc(C2CCCCC2)cc1)(c1ccc(O)c(-c2ccccc2O)c1)c1ccc(O)c(-c2ccccc2O)c1 ; CC(c1ccc(O)cc1)(c1ccc(O)cc1)c1ccc(-c2ccccc2)cc1 ; CC(c1ccc(O)cc1)(c1ccc(O)cc1)c1ccc(C2CCCCC2)cc1]

### Synthesis Examples 20 and 21

Each target product was obtained as in Synthesis Example 10 except that raw materials, 2-naphthol and 4-acetylbiphenyl, were changed to the raw material 1 and the raw material 2 in Table 5; 1.5 mL of water, 73 mg (0.35 mmol) of dodecyl mercaptan and 2.3 g (22 mmol) of 37% hydrochloric acid were added; and the reaction temperature was changed to 55° C. Each product was identified by ¹H-NMR. The results are shown in Table 6.

TABLE 5

| Synthesis | | | |
|---|---|---|---|
| Example | Raw material 1 | Raw material 2 | Product |
| 20 | Resorcinol | 4-Acetyl biphenyl | P-3 |
| 21 | Resorcinol | 4′-Cyclohexyl acetophenone | P-4 |

TABLE 6

| Synthesis | Compound | |
|---|---|---|
| Example | name | 1H-NMR |
| 20 | P-3 | δ (ppm) 9.9 (2H, O—H), |
| | | 6.4-7.8 (15H, Ph—H), 2.3 (3H, C—H) |
| 21 | P-4 | δ (ppm) 9.2 (2H, O—H), |
| | | 6.4-7.2 (10H, Ph—H), 2.3 (3H, C—H), |
| | | 1.4-1.9 (10H, C—H2), 2.7 (1H, C—H) |

From the above results, it was confirmed that the products had chemical structures of the following formulae (P-3) and (P-4), respectively.

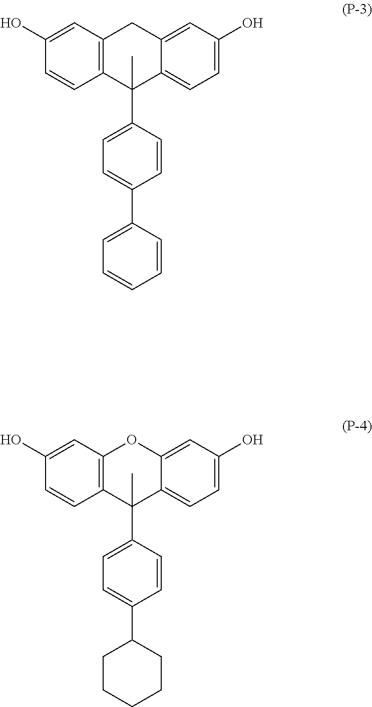

### Example 1

In a clean booth of class 1000, 500 g of a 10% by mass solution having the compound (BisN-1) obtained in Synthesis Example 1 dissolved in propylene glycol monomethyl ether (PGME) was charged into a four-neck flask (bottom outlet type) having a volume of 1000 mL. Subsequently, after removing the air in the tank under reduced pressure, a nitrogen gas was introduced thereinto to return the pressure in the tank to atmospheric pressure. Under nitrogen aeration at 100 mL/min, the oxygen concentration in the tank was adjusted to less than 1%, and the contents in the tank were then heated to 30° C. with stirring. The solution was withdrawn from the bottom outlet valve, passed at a flow rate of 100 mL per minute with a diaphragm pump via a fluororesin pressure tube through a polyamide hollow fiber membrane filter having a nominal filter size of 0.01 μm (trade name: Polyfix nylon series; available from KITZ MICROFILTER CORPORATION), and then collected in a fluororesin vessel. The resulting solution of BisN-1 was analyzed under the following conditions. The oxygen concentration was measured with an oximeter “OM-25MF10” available from AS ONE Corporation, and was maintained at an oxygen concentration of less than 1% until liquid passing was finished (this applied to the following).

### Example 2

The solution was passed as in Example 1 except for using a polyethylene hollow fiber membrane filter having a nominal filter size of 0.01 μm (trade name: Polyfix; available from KITZ MICROFILTER CORPORATION), and the resulting solution of BisN-1 was analyzed under the following conditions.

### Example 3

The solution was passed as in Example 1 except for using a polyamide hollow fiber membrane filter having a nominal filter size of 0.04 μm (trade name: Polyfix; available from KITZ MICROFILTER CORPORATION), and the resulting solution of BisN-1 was analyzed under the following conditions.

### Example 4

The solution was passed as in Example 1 except for using a polyethylene membrane filter having a nominal filter size of 5 nm (trade name: Protego; available from Entegris Japan Co., Ltd.), and the resulting solution of BisN-1 was analyzed under the following conditions.

### Example 5

The solution was passed as in Example 1 except for using a PTFE membrane filter having a nominal filter size of 0.05 μm (trade name: Omnipore; available from Millipore Corporation), and the resulting solution of BisN-1 was analyzed under the following conditions.

### Example 6

The solution was passed as in Example 1 except for using a Zeta Plus filter 40QSH having a nominal filter size of 0.2 μm (having an ion exchange capacity; available from 3M Company), and the resulting solution of BisN-1 was analyzed under the following conditions.

### Example 7

The solution was passed as in Example 1 except for using a Zeta Plus filter 020GN having a nominal filter size of 0.2 μm (having an ion exchange capacity; available from 3M Company), and the resulting solution of BisN-1 was analyzed under the following conditions.

### Example 8

The solution was passed as in Example 1 except that the compound (BisN-2) obtained in Synthesis Example 2 was used instead of the compound (BisN-1) in Example 1, and the resulting solution of BisN-2 was analyzed under the following conditions.

### Example 9

The solution was passed as in Example 1 except that the resin (RBisN-2) obtained in Synthesis Example 3 was used instead of the compound (BisN-1) in Example 1, and the resulting solution of RBisN-2 was analyzed under the following conditions.

### Example 10

The solution was passed through the filter as in Example 1 except that the compound (CH—BisN) obtained in Synthesis Example 4 was used instead of the compound (BisN-1) in Example 1, and the resulting solution of CH-BisN was analyzed under the following conditions.

### Example 11

The solution was passed through the filter as in Example 1 except that the compound (CAX-1) obtained in Synthesis Example 5 was used instead of the compound (BisN-1) in Example 1, and the resulting solution of CAX-1 was analyzed under the following conditions.

### Example 12

The solution was passed through the filter as in Example 1 except that the compound (BiF-1) obtained in Synthesis Example 6 was used instead of the compound (BisN-1) in Example 1, and the resulting solution of BiF-1 was analyzed under the following conditions.

### Example 13

The solution was passed through the filter as in Example 1 except that the compound (BiF-1-1) obtained in Synthesis Example 7 was used instead of the compound (BisN-1) in Example 1, and the resulting solution of BiF-1-1 was analyzed under the following conditions.

### Example 14

The solution was passed through the filter as in Example 1 except that the compound (P-6) obtained in Synthesis Example 8 was used instead of the compound (BisN-1) in Example 1, and the resulting solution of P-6 was analyzed under the following conditions.

### Example 15

The solution was passed through the filter as in Example 1 except that the compound (P-7) obtained in Synthesis Example 9 was used instead of the compound (BisN-1) in Example 1, and the resulting solution of P-7 was analyzed under the following conditions.

### Example 16

The solution was passed through the filter as in Example 1 except that the compound (BiN-1) obtained in Synthesis Example 10 was used instead of the compound (BisN-1) in Example 1, and the resulting solution of BiN-1 was analyzed under the following conditions.

### Example 17

The solution was passed through the filter as in Example 1 except that the compound (BiP-1) obtained in Synthesis Example 11 was used instead of the compound (BisN-1) in Example 1, and the resulting solution of BiP-1 was analyzed under the following conditions.

### Example 18

The solution was passed through the filter as in Example 1 except that the compound (BiN-2) obtained in Synthesis Example 12 was used instead of the compound (BisN-1) in Example 1, and the resulting solution of BiN-2 was analyzed under the following conditions.

### Example 19

The solution was passed through the filter as in Example 1 except that the compound (BiN-3) obtained in Synthesis Example 13 was used instead of the compound (BisN-1) in Example 1, and the resulting solution of BiN-3 was analyzed under the following conditions.

### Example 20

The solution was passed through the filter as in Example 1 except that the compound (BiN-4) obtained in Synthesis Example 14 was used instead of the compound (BisN-1) in Example 1, and the resulting solution of BiN-4 was analyzed under the following conditions.

### Example 21

The solution was passed through the filter as in Example 1 except that the compound (BiP-2) obtained in Synthesis Example 15 was used instead of the compound (BisN-1) in Example 1, and the resulting solution of BiP-2 was analyzed under the following conditions.

### Example 22

The solution was passed through the filter as in Example 1 except that the compound (BiP-3) obtained in Synthesis Example 16 was used instead of the compound (BisN-1) in Example 1, and the resulting solution of BiP-3 was analyzed under the following conditions.

### Example 23

The solution was passed through the filter as in Example 1 except that the compound (BiP-4) obtained in Synthesis Example 17 was used instead of the compound (BisN-1) in Example 1, and the resulting solution of BiP-4 was analyzed under the following conditions.

### Example 24

The solution was passed through the filter as in Example 1 except that the compound (P-1) obtained in Synthesis Example 18 was used instead of the compound (BisN-1) in Example 1, and the resulting solution of P-1 was analyzed under the following conditions.

### Example 25

The solution was passed through the filter as in Example 1 except that the compound (P-2) obtained in Synthesis Example 19 was used instead of the compound (BisN-1) in Example 1, and the resulting solution of P-2 was analyzed under the following conditions.

### Example 26

The solution was passed through the filter as in Example 1 except that the compound (P-3) obtained in Synthesis Example 20 was used instead of the compound (BisN-1) in Example 1, and the resulting solution of P-3 was analyzed under the following conditions.

### Example 27

The solution was passed through the filter as in Example 1 except that the compound (P-4) obtained in Synthesis Example 21 was used instead of the compound (BisN-1) in Example 1, and the resulting solution of P-4 was analyzed under the following conditions.

### (Example 28) without Nitrogen Gas Replacement

In a clean booth of class 1000, 500 g of a 2.5% by mass solution having BisN-1 dissolved in PGME was charged into a four-neck flask (bottom outlet type) having a volume of 1000 mL, and heated to 30° C. with stirring. The oxygen concentration was measured with an oximeter “OM-25MF10” available from AS ONE Corporation, and as a result, it was 20.8%. The solution of BisN-1 was withdrawn from the bottom outlet valve, passed at a flow rate of 100 mL per minute with a diaphragm pump via a fluororesin pressure tube through a polyamide hollow fiber membrane filter having a nominal filter size of 0.01 μm (trade name: Polyfix nylon series; available from KITZ MICROFILTER CORPORATION). The resulting solution of BisN-1 was analyzed under the following conditions.

### (Comparative Example 1) without Passing Liquid Through Filter

In a clean booth of class 1000, 500 g of a solution having BisN-1 dissolved in PGME (concentration: 2.5% by mass) was charged into a four-neck flask (bottom outlet type) having a volume of 1000 mL. Subsequently, after removing the air in the tank under reduced pressure, a nitrogen gas was introduced thereinto to return the pressure in the tank to atmospheric pressure. Under nitrogen aeration at 100 mL/min, the contents in the tank were then heated to 30° C. with stirring. The solution of BisN-1 was withdrawn from the bottom outlet valve, and collected at a flow rate of 100 mL per minute with a diaphragm pump via a fluororesin pressure tube in a fluororesin vessel. The collected solution of BisN-1 was analyzed under the following conditions.

The metal contents and organic purity of the various PGME solutions obtained in Examples 1 to 28 and Comparative Example 1 were measured. The measurement results are shown in Table 7. Each measurement was made under the following conditions with the following device.

### [Measurement of Contents of Various Metals]

The contents of metals in various PGME solutions were measured under the following measurement conditions with ICP-MS.

Device: ELAN DRC II (available from Perkin Elmer)

Temperature: 25° C.

Environment: Clean room of class 100

### [Measurement of Organic Purity]

The organic purity in various PGME solutions was measured under the following measurement conditions by high-performance liquid chromatography.

Device: GL-7400 (available from Hitachi)

Column: X-BRIDE C18

Eluent: acetonitrile/water

Temperature: 40° C.

As used herein, the term “organic purity” means the proportion (% by mass) of the mass of a compound or resin (for example, BisN-1 in Example 1) to the total mass of the organic compounds dissolved in the PGME solution.

TABLE 7

| | |
|---|---|
| Metal content (ppb) | Organic |

| | Na | Fe | Cr | Sn | purity (%) |
|---|---|---|---|---|---|
| Example 1 | <0.2 | <0.2 | <0.2 | <0.2 | 99.3 |
| Example 2 | <0.2 | <0.2 | 0.8 | 0.5 | 99.3 |
| Example 3 | <0.2 | <0.2 | <0.2 | <0.2 | 99.2 |
| Example 4 | <0.2 | 2 | 2 | 1 | 99.3 |
| Example 5 | <0.2 | 2 | 3 | 1 | 99.3 |
| Example 6 | <0.2 | <0.2 | 1 | 1 | 99.1 |
| Example 7 | <0.2 | <0.2 | 1 | 1 | 99.3 |
| Example 8 | <0.2 | <0.2 | <0.2 | <0.2 | 99.2 |
| Example 9 | <0.2 | <0.2 | <0.2 | <0.2 | 98.9 |
| Example 10 | <0.2 | <0.2 | <0.2 | <0.2 | 99.2 |
| Example 11 | <0.2 | <0.2 | <0.2 | <0.2 | 99.5 |
| Example 12 | <0.2 | <0.2 | <0.2 | <0.2 | 98.8 |
| Example 13 | <0.2 | <0.2 | <0.2 | <0.2 | 99.2 |
| Example 14 | <0.2 | <0.2 | <0.2 | <0.2 | 99.2 |
| Example 15 | <0.2 | <0.2 | <0.2 | <0.2 | 98.5 |
| Example 16 | <0.2 | <0.2 | <0.2 | <0.2 | 99.1 |
| Example 17 | <0.2 | <0.2 | <0.2 | <0.2 | 99.6 |
| Example 18 | <0.2 | <0.2 | <0.2 | <0.2 | 99.0 |
| Example 19 | <0.2 | <0.2 | <0.2 | <0.2 | 98.9 |
| Example 20 | <0.2 | <0.2 | <0.2 | <0.2 | 99.2 |
| Example 21 | <0.2 | <0.2 | <0.2 | <0.2 | 98.7 |
| Example 22 | <0.2 | <0.2 | <0.2 | <0.2 | 99.2 |
| Example 23 | <0.2 | <0.2 | <0.2 | <0.2 | 99.0 |
| Example 24 | <0.2 | <0.2 | <0.2 | <0.2 | 99.1 |
| Example 25 | <0.2 | <0.2 | <0.2 | <0.2 | 98.8 |
| Example 26 | <0.2 | <0.2 | <0.2 | <0.2 | 98.7 |
| Example 37 | <0.2 | <0.2 | <0.2 | <0.2 | 99.1 |
| Example 28 | <0.2 | <0.2 | <0.2 | <0.2 | 97.5 |
| Comparative | >99 | 60 | 80 | 70 | 99.3 |
| Example 1 | | | | | |

Table 7 shows that according to the purification method according to the present embodiment, the metal content in a compound/resin having a given structure can be reduced. That is, it can be seen that the method for producing a composition according to the present embodiment can provide a composition containing the above-described compound/resin wherein the contents of metals as impurities are reduced.

The present application claims the priority based on Japanese Patent Application (Japanese Patent Application No. 2017-037388) filed on Feb. 28, 2017, the contents of which are incorporated herein by reference.

### INDUSTRIAL APPLICABILITY

According to the present invention, the material having a specific structure, which has a metal content significantly reduced, can be industrially advantageously produced.

1. A method for purifying a material, the method comprising:
a step of preparing a solution comprising a solvent and at least one material selected from the group consisting of a compound represented by the following formula (1A) and a resin having a structure represented by the following formula (2A); and
a step of purification in which the solution is passed through a filter: 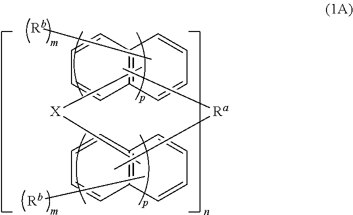
wherein, X represents an oxygen atom, a sulfur atom, a single bond, or non-crosslinked state; R^{a} represents a 2n-valent group having 1 to 60 carbon atoms or a single bond; each R^{b} independently represents an optionally substituted alkyl group having 1 to 40 carbon atoms, an optionally substituted aryl group having 6 to 40 carbon atoms, an optionally substituted alkenyl group having 2 to 40 carbon atoms, an optionally substituted alkoxy group having 1 to 40 carbon atoms, a halogen atom, a thiol group or a hydroxyl group; each m is independently an integer of 0 to 9; n is an integer of 1 to 4; and each p is independently an integer of 0 to 2; provided that at least one R^{b} represents a group comprising one selected from a hydroxyl group and a thiol group, and all m cannot be 0 at the same time; 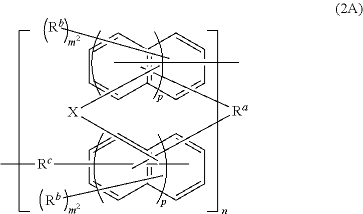
wherein, X, R^{a}, R^{b}, n and p are the same as defined in the formula (1A); R^{c} represents a single bond or an alkylene group having 1 to 40 carbon atoms; each m² is independently an integer of 0 to 8; provided that at least one R^{b} represents a group comprising one or more selected from a hydroxyl group and a thiol group, and all m² cannot be 0 at the same time.

2. The method for purifying the material according to claim 1, wherein the purification is performed in an atmosphere with an oxygen concentration of less than 20%.

3. The method for purifying the material according to claim 1, wherein the filter has a nominal pore size of 0.2 μm or less.

4. The method for purifying the material according to claim 1, wherein the filter is one or more selected from the group consisting of a hollow fiber membrane filter, a membrane filter and a pleated membrane filter.

5. The method for purifying the material according to claim 1, wherein the filter is made of one or more filter media selected from the group consisting of a polyamide, a polyolefin resin and a fluorocarbon resin.

6. The method for purifying the material according to claim 1, wherein the filter comprises an ion exchanger.

7. The method for purifying the material according to claim 1, wherein the filter comprises a material having a zeta potential.

8. The method for purifying the material according to claim 1, wherein the solvent is one or more selected from the group consisting of ethyl acetate, butyl acetate, methyl isobutyl ketone, propylene glycol monomethyl ether, propylene glycol monomethyl ether acetate, cyclopentanone and cyclohexanone.

9. The method for purifying the material according to claim 1, wherein a content of chromium among metals comprised in the solution after the purification is 50 ppb or less based on a mass of the material.

10. The method for purifying the material according to claim 1, wherein the compound represented by the formula (1A) and the resin having a structure represented by the formula (2A) are a compound represented by the following formula (1A′) and a resin having a structure represented by the following formula (2A′), respectively: 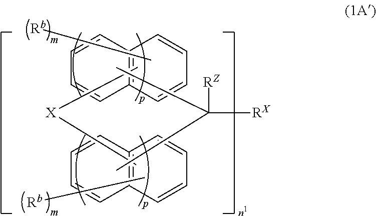
wherein, R^{b}, X, m and p are the same as defined in the formula (1A); R^{x} represents an n-valent group having 1 to 40 carbon atoms or a single bond; R^{z} represents a hydrogen atom, an alkyl group having 1 to 30 carbon atoms or an aryl group having 6 to 30 carbon atoms; and n¹ is an integer of 1 to 4; 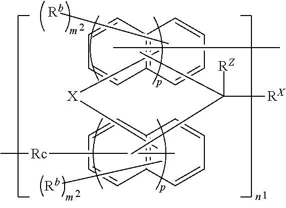
wherein, R^{b}, X, m² and p are the same as defined in the formula (2A); and R^{x}, R^{z} and n¹ are the same as defined in the formula (1A′).

11. The method for purifying the material according to claim 1, wherein the compound represented by the formula (1A) is a compound represented by the formula (1): 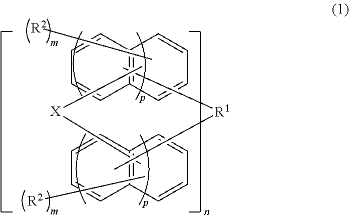
wherein, X, m, n and p are the same as defined in the formula (1A); R¹ is the same as R^{a} defined in the formula (1A); and each R² independently represents an alkyl group having 1 to 40 carbon atoms, an aryl group having 6 to 40 carbon atoms, an alkenyl group having 2 to 40 carbon atoms, an alkoxy group having 1 to 40 carbon atoms, a halogen atom, a thiol group or a hydroxyl group; provided that at least one R² represents one selected from a hydroxyl group and a thiol group, and all m cannot be 0 at the same time.

12. The method for purifying the material according to claim 11, wherein the compound represented by the formula (1) is a compound represented by the following formula (1-1): 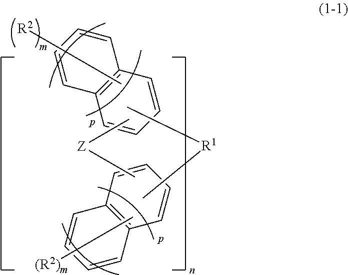
wherein, Z represents an oxygen atom or a sulfur atom; R¹, R², m, p and n are the same as defined in the formula (1); provided that at least one R² represents one selected from a hydroxyl group and a thiol group, and all m cannot be 0 at the same time.

13. The method for purifying the material according to claim 12, wherein the compound represented by the formula (1-1) is a compound represented by the following formula (1-2): 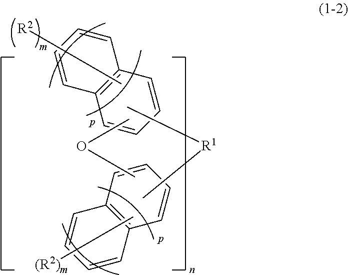
wherein, R¹, R², m, p and n are the same as defined in the formula (1); provided that at least one R² represents one selected from a hydroxyl group and a thiol group, and all m cannot be 0 at the same time.

14. The method for purifying the material according to claim 13, wherein the compound represented by the formula (1-2) is a compound represented by the following formula (1-3): 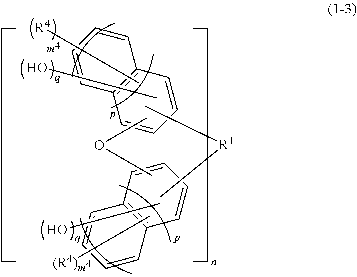
wherein, R¹, p and n are the same as defined in the formula (1); each R⁴ independently represents an alkyl group having 1 to 40 carbon atoms, an aryl group having 6 to 40 carbon atoms, an alkenyl group having 2 to 40 carbon atoms, an alkoxy group having 1 to 40 carbon atoms, a halogen atom or a thiol group; each m⁴ is independently an integer of 0 to 8; and each q is independently an integer of 0 to 8; provided that all q cannot be 0 at the same time.

15. The method for purifying the material according to claim 14, wherein the compound represented by the formula (1-3) is a compound represented by the following formula (1-4): 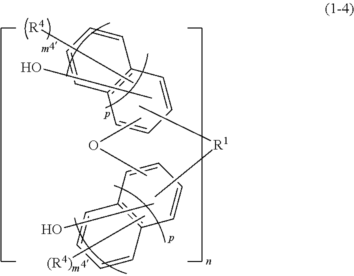
wherein, R¹, p and n are the same as defined in the formula (1); R⁴ is the same as defined in the formula (1-3); and each m^{4′} is independently an integer of 0 to 7.

16. The method for purifying the material according to claim 15, wherein the compound represented by the formula (1-4) is a compound represented by the following formula (1-5): 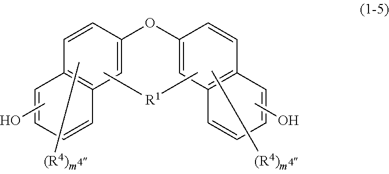
wherein, R¹ is the same as defined in the formula (1); R⁴ is the same as defined in the formula (1-3); and each m^{4″} is independently an integer of 0 to 5.

17. The method for purifying the material according to claim 1, wherein the compound represented by the formula (1A) is a compound represented by the following formula (3): 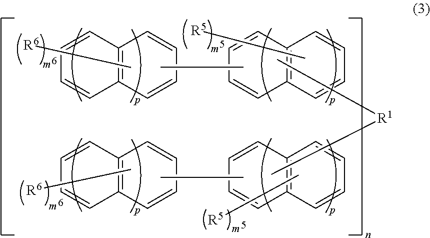
wherein, R¹ is the same as R^{a} defined in the formula (1A); n and p are the same as defined in the formula (1A); R⁵ and R⁶ each independently represent an alkyl group having 1 to 40 carbon atoms, an aryl group having 6 to 40 carbon atoms, an alkenyl group having 2 to 40 carbon atoms, an alkoxy group having 1 to 40 carbon atoms, a halogen atom, a thiol group or a hydroxyl group; each m⁵ is independently an integer of 0 to 8; and each m⁶ is independently an integer of 0 to 9; provided that at least one selected from R⁵ and R⁶ represents one selected from a hydroxyl group and a thiol group, and all of m⁵ and m⁶ cannot be 0 at the same time.

18. The method for purifying the material according to claim 17, wherein the compound represented by the formula (3) is a compound represented by the following formula (3-1): 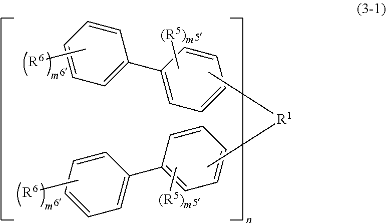
wherein, R¹, R⁵, R⁶ and n are the same as defined in the formula (3); each m^{5′} is independently an integer of 0 to 4; and each m^{6′} is independently an integer of 0 to 5; provided that at least one selected from R⁵ and R⁶ represents one selected from a hydroxyl group and a thiol group, and all of m⁵ and m^{6′} cannot be 0 at the same time.

19. The method for purifying according to claim 18, wherein the compound represented by the formula (3-1) is a compound represented by the following formula (3-2): 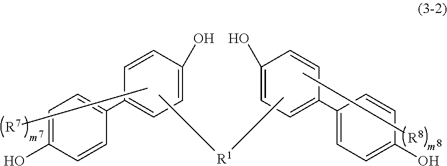
wherein, R¹ is the same as defined in the formula (3); R⁷ and R⁸ each independently represent an alkyl group having 1 to 40 carbon atoms, an aryl group having 6 to 40 carbon atoms, an alkenyl group having 2 to 40 carbon atoms, an alkoxy group having 1 to 40 carbon atoms, a halogen atom, a thiol group or a hydroxyl group; m⁷ and m⁸ each are independently an integer of 0 to 7.

20. The method for purifying the material according to claim 1, wherein the resin having a structure represented by the formula (2A) is a resin having a structure represented by the following formula (2): 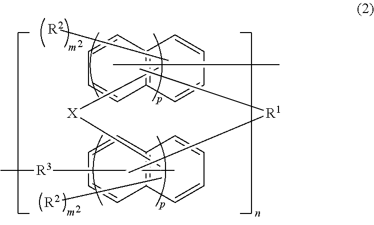
wherein, X, R¹, R², n and p are the same as defined in the formula (1); R³ is the same as R^{c} defined in the formula (2A); and m² is the same as defined in the formula (2A); provided that at least one R² represents one selected from a hydroxyl group and a thiol group, and all m² cannot be 0 at the same time.

21. The method for purifying the material according to claim 20, wherein the resin having a structure represented by the formula (2) is a resin having a structure represented by the following formula (2-1): 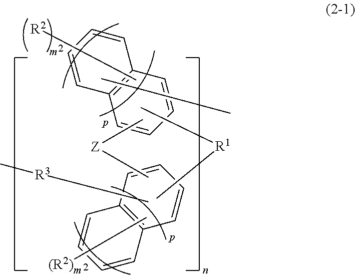
wherein, Z is the same as defined in the formula (1-1); R¹, R², R³, m², p and n are the same as defined in the formula (2); provided that at least one R² represents one selected from a hydroxyl group and a thiol group, and all m² cannot be 0 at the same time.

22. The method for purifying the material according to claim 1, wherein the resin having a structure represented by the formula (2A) is a resin having a structure represented by the following formula (4): 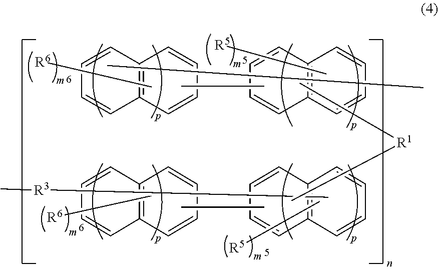
wherein, R¹, R⁵, R⁶, m⁵, m⁶, p and n are the same as defined in the formula (3); and R³ is the same as defined in the formula (2); provided that at least one selected from R⁵ and R⁶ represents one selected from a hydroxyl group and a thiol group, and all of m⁵ and m⁶ cannot be 0 at the same time.

23. A method for producing a composition comprising at least one material selected from the group consisting of a compound represented by the following formula (1A) and a resin having a structure represented by the following formula (2A), 99 ppb or less of Na, less than 60 ppb of Fe, less than 80 ppb of Cr and less than 70 ppb of Sn, the method comprising:
a step of preparing a solution comprising a solvent and a precursor composition comprising the material and more than 99 ppb of Na, 60 ppb or more of Fe, 80 ppb or more of Cr and 70 ppb or more of Sn; and
a step of passing the solution through a filter to thereby reduce contents of Na, Fe, Cr and Sn in the solution to 99 ppb or less, less than 60 ppb, less than 80 ppb and less than 70 ppb, respectively: 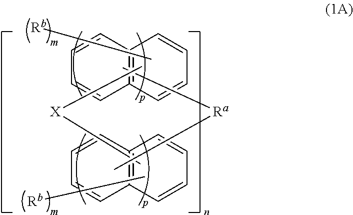
wherein, X represents an oxygen atom, a sulfur atom, a single bond, or non-crosslinked state; R^{a} represents a 2n-valent group having 1 to 60 carbon atoms or a single bond; each R^{b} independently represents an optionally substituted alkyl group having 1 to 40 carbon atoms, an optionally substituted aryl group having 6 to 40 carbon atoms, an optionally substituted alkenyl group having 2 to 40 carbon atoms, an optionally substituted alkoxy group having 1 to 40 carbon atoms, a halogen atom, a thiol group or a hydroxyl group; each m is independently an integer of 0 to 9; n is an integer of 1 to 4; and each p is independently an integer of 0 to 2; provided that at least one R^{b} represents a group comprising one selected from a hydroxyl group and a thiol group, and all m cannot be 0 at the same time; 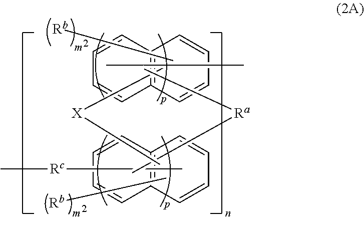
wherein, X, R^{a}, R^{b}, n and p are the same as defined in the formula (1A); R^{c} represents a single bond or an alkylene group having 1 to 40 carbon atoms; each m² is independently an integer of 0 to 8; provided that at least one R^{b} represents a group comprising one or more selected from a hydroxyl group and a thiol group, and all m² cannot be 0 at the same time.
